(19) United States
(12) Patent Application Publication
(10) Pub. No.: US 2026/0146074 A1
(43) Pub. Date: May 28, 2026

(54) TAG72 TARGETED CHIMERIC ANTIGEN RECEPTOR MODIFIED T CELLS FOR TREATMENT OF TAG72-POSITIVE TUMORS

(71) Applicant: City of Hope, Duarte, CA (US)

(72) Inventors: Saul J. Priceman, Valley Village, CA (US); John P. Murad, Diamond Bar, CA (US); Stephen J. Forman, Duarte, CA (US); John E. Shively, Arcadia, CA (US); Paul Yazaki, Glendale, CA (US); David Colcher, Duarte, CA (US); Anna Kozlowska, Duarte, CA (US); Hee Jun Lee, Diamond Bar, CA (US)

(21) Appl. No.: 19/178,637

(22) Filed: Apr. 14, 2025

Related U.S. Application Data
(63) Continuation of application No. 17/264,911, now Pat. No. 12,281,152.
(60) Provisional application No. 62/713,485, filed on Aug. 1, 2018.

(51) Int. Cl. C07K 14/725; A61K 40/11; A61K 40/31; A61K 40/42; A61P 35/00; C07K 14/705; C07K 14/715; C07K 14/73; C07K 16/30; C12N 5/0783

(52) U.S. Cl. CPC C07K 14/7051; A61K 40/11; A61K 40/31; A61K 40/4256; A61K 40/4257; A61P 35/00; C07K 14/70514; C07K 14/70521; C07K 14/7151; C07K 16/3092; C12N 5/0636; A61K 2239/31; A61K 2239/59; C07K 2317/53; C07K 2317/622; C07K 2319/02; C07K 2319/03; C07K 2319/33; C12N 2501/20

(57) ABSTRACT

Chimeric antigen receptors targeted to TAG72 and the use thereof to treat ovarian cancer and other cancers are described.

### CROSS-REFERENCE TO RELATED APPLICATIONS

This application is a continuation of U.S. patent application Ser. No. 17/264,911, filed on Feb. 1, 2021, which is a U.S. National Phase Application under 35 U.S.C. § 371 of International Application No. PCT/US2019/044751, filed on Aug. 1, 2019, which claims priority to and the benefit of U.S. Provisional Application No. 62/713,485, filed on Aug. 1, 2018. The entire contents of the foregoing are incorporated herein by reference.

### SEQUENCE LISTING

This application contains a Sequence Listing that has been submitted electronically as an XML file named 40056-0043002_SL_ST26.xml. The XML file, created on Apr. 10, 2025, is 44,069 bytes in size. The material in the XML file is hereby incorporated by reference in its entirety.

### TECHNICAL FIELD

This disclosure concerns tumor-associated glycoprotein 72 (TAG72)-specific chimeric antigen receptor (CAR)-engineered T cells, methods of formulating, and methods of use as anti-cancer agents selective against TAG72-positive cells.

### BACKGROUND

Chimeric Antigen Receptor (CAR)-engineered T cell therapy in patients with CD19+ B-cell malignancies have demonstrated impressive clinical responses, which have recently resulted in two landmark FDA approvals for patients with leukemia and lymphoma (Maude S L, Teachey D T, Porter D L, Grupp S A. CD19-targeted chimeric antigen receptor T-cell therapy for acute lymphoblastic leukemia. Blood. 2015; 125(26):4017-23; Jain M D, Davila M L. Concise Review: Emerging Principles from the Clinical Application of Chimeric Antigen Receptor T Cell Therapies for B Cell Malignancies. Stem cells. 2018; 36(1):36-44). These studies have shown that CAR T cells can be optimized to induce durable and complete responses in cancer patients, even under conditions of highly refractory disease. Major obstacles in developing effective CAR T cell therapies for solid cancers is avoiding off-tumor on-target toxicity due to the lack of truly restricted tumor antigens, as well as achieving durable responses that are limited by T cell persistence and tumor trafficking (Priceman S J, Forman S J, Brown C E. Smart CARs engineered for cancer immunotherapy. Current opinion in oncology. 2015; 27(6):466-74; Chen N, Li X, Chintala N K, Tano Z E, Adusumilli P S. Driving CARs on the uneven road of antigen heterogeneity in solid tumors. Current opinion in immunology. 2018; 51:103-10). To date, the majority of tumor antigens for directing specificity of CAR T cells have targeted over-expressed proteins, including but not limited to mesothelin, PSMA, PSCA, HER2/neu, EGFR, and IL13Rα2 (Priceman S J, Forman S J, Brown C E. Smart CARs engineered for cancer immunotherapy. Current opinion in oncology. 2015; 27(6):466-74; Yong C S M, Dardalhon V, Devaud C, Taylor N, Darcy P K, Kershaw M H. CAR T-cell therapy of solid tumors. Immunology and cell biology. 2017; 9 5(4):356-63). While the field is still evolving, the clinical efficacy of CAR T cells targeting these proteins in solid tumors have been somewhat limited (Castellarin M, Watanabe K, June C H, Kloss C C, Posey A D, Jr. Driving cars to the clinic for solid tumors. Gene therapy. 2018. Epub 2018/06/09.), and identification of additional targets as well as addressing limited T cell durability continue to be outstanding problems limiting the success of CAR T cell therapies.

Aberrant glycosylation of cell surface proteins on tumors have long been implicated in tumor development, and have unique glycoprotein signatures that are attractive targets for immunotherapy, including CAR T cells (Steentoft C, Migliorini D, King T R, Mandel U, June C H, Posey A D, Jr. Glycan-Directed Car-T Cells. Glycobiology. 2018. Epub 2018/01/26; RodrIguez E, Schetters S T T, van Kooyk Y. The tumour glyco-code as a novel immune checkpoint for immunotherapy. Nature reviews Immunology. 2018; 18(3):204-11. Epub 2018/02/06). Multiple cancer types including colon, breast, pancreas, and ovarian, are known to over-express glycoproteins, including the mucins MUC16 and MUC1, and tumor associated glycoprotein-72 (TAG72) (Hollingsworth M A, Swanson B J. Mucins in cancer: protection and control of the cell surface. Nature reviews Cancer. 2004; 4(1):45-60. Epub 2003/12/19), that differentiate them from normal epithelia. TAG72 is a high molecular weight mucin with large amounts of O-glycosidic linkages to serine and threonine residues (Julien S, Videira P A, Delannoy P. Sialyl-tn in cancer: (how) did we miss the target? Biomolecules. 2012; 2(4):435-66. Epub 2012/01/01). High expression of TAG72, MUC1, and MUC16 has been shown in ovarian cancer patient tissue samples, with nearly 100-percent of ovarian cancers identified with simultaneous staining of the three antigens (Chauhan S C, Vinayek N, Maher D M, Bell M C, Dunham K A, Koch M D, Lio Y, Jaggi M. Combined staining of TAG72, MUC1, and CA125 improves labeling sensitivity in ovarian cancer: antigens for multi-targeted antibody-guided therapy. The journal of histochemistry and cytochemistry. 2007; 55(8):867-75). Importantly, approximately 90-percent of epithelial ovarian cancers are TAG72 positive, indicating its abundance across multiple histological subtypes of ovarian cancer.

Several monoclonal antibodies specific to the tumor-associated sialyl Tn antigen (STn antigen) of TAG72 have been developed, including the well-studied clone CC49 (Muraro R, Kuroki M, Wunderlich D, Poole D J, Colcher D, Thor A, Greiner J W, Simpson J F, Molinolo A, Noguchi P, et al. Generation and characterization of B72.3 second generation monoclonal antibodies reactive with the tumor-associated glycoprotein 72 antigen. Cancer research. 1988; 48(16):4588-96). CC49 has been subsequently utilized in multiple pre-clinical and clinical investigations using diagnostic imaging and radiotherapy and also involved in multiple attempts of antibody humanization (Cheng K T. Radioiodinated anti-TAG72 CC49 Fab′ antibody fragment. Molecular Imaging and Contrast Agent Database (MICAD). Bethesda MD2004; Pavlinkova G, Booth B J, Batra S K, Colcher D. Radioimmunotherapy of human colon cancer xenografts using a dimeric single-chain Fv antibody construct. Clinical cancer research: an official journal of the American Association for Cancer Research. 1999; 5(9):2613-9; Kashmiri S V, Shu L, Padlan E A, Milenic D E, Schlom J, Hand P H. Generation, characterization, and in vivo studies of humanized anticarcinoma antibody CC49. Hybridoma. 1995; 14(5):461-73; De Pascalis R, Gonzales N R, Padlan E A, Schuck P, Batra S K, Schlom J, Kashmiri S V. In vitro affinity maturation of a specificity-determining region-grafted humanized anticarcinoma antibody: isolation and characterization of minimally immunogenic high-affinity variants. Clinical cancer research: an official journal of the American Association for Cancer Research. 2003; 9(15):5521-31; Gonzales N R, Padlan E A, De Pascalis R, Schuck P, Schlom J, Kashmiri S V. Minimizing immunogenicity of the SDR-grafted humanized antibody CC49 by genetic manipulation of the framework residues. Molecular immunology. 2003; 40(6):337-49; Pavlinkova G, Colcher D, Booth B J, Goel A, Wittel U A, Batra S K. Effects of humanization and gene shuffling on immunogenicity and antigen binding of anti-TAG72 single-chain Fvs. International journal of cancer. 2001; 94(5):717-26; Hege K M, Bergsland E K, Fisher G A, Nemunaitis J J, Warren R S, McArthur J G, Lin A A, Schlom J, June C H, Sherwin S A. Safety, tumor trafficking and immunogenicity of chimeric antigen receptor (CAR)-T cells specific for TAG72 in colorectal cancer. Journal for immunotherapy of cancer. 2017; 5:22).

### SUMMARY

Described herein are methods for using TAG72 targeted CAR T cells to treat a variety of cancers, for example, ovarian cancer.

Described herein is a nucleic acid molecule comprising a nucleotide sequence encoding a chimeric antigen receptor (CAR), wherein the chimeric antigen receptor comprises: an scFv targeting Tag-72, a spacer, a transmembrane domain, a 41-BB co-stimulatory domain or CD28 co-stimulatory domain, and a CD3 ζ signaling domain.

In various embodiments: the transmembrane domain is selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications; the spacer comprises 20-150 amino acids and is located between the scFv and the transmembrane domain; the transmembrane domain is a CD4 transmembrane domain or variant thereof having 1-5 amino acid modifications; the transmembrane domain is a CD4 transmembrane domain; the chimeric antigen receptor comprises a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications; the spacer region comprises an amino acid sequence selected from the group consisting of SEQ ID NOs: 2-12 or a variant thereof having 1-5 amino acid modifications; the spacer comprises an IgG hinge region; the spacer comprises 10-50 amino acids; the 4-1BB costimulatory domain comprises the amino acid sequence of SEQ ID NO: 24 or a variant thereof having 1-5 amino acid modifications; the CD3ζ signaling domain comprises the amino acid sequence of SEQ ID NO:21; a linker of 3 to 15 amino acids is located between the 4-1BB costimulatory domain and the CD3 ζ signaling domain or variant thereof; the CAR comprises the amino acid sequence of SEQ ID NO: 29 or a variant thereof having 1-5 amino acid modifications; the scFv comprises the amino acid sequence of SEQ ID NO:1, SEQ ID NO:33 or SEQ ID NO:34.

Also disclosed herein is: a viral vector comprising a nucleic acid molecule described herein; a population of human T cells (e.g., a population comprising central memory T cells) transduced by a vector comprising a nucleic acid molecule described herein.

Also described herein is a method of treating solid tumor in a patient comprising administering a population of autologous or allogeneic human T cells transduced by a vector comprising a nucleic acid molecule described herein, wherein the solid tumor comprises cells expressing Tag-72. In various embodiments: the chimeric antigen receptor is administered locally or systemically; the TAG72-expressing cells are ovarian cancer cells; and the chimeric antigen receptor is administered by single or repeat dosing.

In various embodiments: the chimeric antigen receptor comprises: a TAG72 scFv (e.g., an scFv comprising the amino acid sequence: QVQLVQSGAEVVKPGASVKISCKASGYTFTDHAIHWVKQNPGQRLEWIGYFSPGNDDF KYNERFKGKATLTADTSASTAYVELSSLRSEDTAVYFCTRSLNMAYWGQGTLVTVSSG STSGGGSGGGSGGGGSSDIVMSQSPDSLAVSLGERVTLNCKSSQSLLYSGNQKNYLAW YQQKPGQSPKLLIYWASARESGVPDRFSGSGSGTDFTLTISSVQAEDVAVYYCQQYYSY PLTFGAGTKLELK (SEQ ID NO:1) with up to 5 or up to 10 single amino acid substitutions).

In various embodiments: the chimeric antigen receptor comprises: a TAG72 V15 scFv (e.g., an scFv comprising the amino acid sequence: QVQLVQSGAEVVKPGASVKISCKASGYTFTDHAIHWVKQNPGQRLEWIGYFSPGNDDF KYSQKFQGKATLTADTSASTAYVELSSLRSEDTAVYFCTRSLNMAYWGQGTLVTVSSG STSGGGSGGGSGGGGSSDIVMSQSPDSLAVSLGERVTLNCKSSQSVLYSSNSKNYLAWY QQKPGQSPKLLIYWASTRESGVPDRFSGSGSGTDFTLTISSVQAEDVAVYYCQQYYSYP LSFGAGTKLELK (SEQ ID NO:33) with up to 5 or up to 10 single amino acid substitutions).

In various embodiments: the chimeric antigen receptor comprises: a TAG72 V59_V15 scFv (e.g., an scFv comprising the amino acid sequence: QVQLVQSGAEVKKPGASVKVSCKASGYTFTDHAIHWVRQAPGQRLEWMGYFSPGNDD FKYSQKFQGRVTITADTSASTAYMELSSLRSEDTAVYFCTRSLNMAYWGQGTLVTVSS GSTSGGGSGGGSGGGGSSDIVMTQSPDSLAVSLGERATINCKSSQSLLYSSNSKNYLAW YQQKPGQPPKLLIYWASTRESGVPDRFSGSGSGTDFTLTISSLQAEDVAVYYCQQPYSYP LSFGAGTKLELK (SEQ ID NO:34) with up to 5 or up to 10 single amino acid substitutions).

Also described a T cells harboring a vector expressing the CAR. In various embodiments: at least 20%, 30%, or 40% of the transduced human T cells are central memory T cells; at least 30% of the transduced human T cells are CD4+ and CD62L+ or CD8+ and CD62L+; the population of human T cells are autologous to the patient; and the population of human T cells are allogenic to the patient.

### TAG72 Targeted CAR

The TAG72 targeted CAR described herein include a TAG72 targeting scFv (e.g., an scFv comprising the amino acid sequence:

(SEQ ID NO: 1)
QVQLVQSGAEVVKPGASVKISCKASGYTFTDHAIHWVKQNPGQRLEWIG
YFSPGNDDFKYNERFKGKATLTADTSASTAYVELSSLRSEDTAVYFCTR
SLNMAYWGQGTLVTVSSGSTSGGGSGGGSGGGGSSDIVMSQSPDSLAVS
LGERVTLNCKSSQSLLYSGNQKNYLAWYQQKPGQSPKLLIYWASARESG
VPDRFSGSGSGTDFTLTISSVQAEDVAVYYCQQYYSYPLTFGAGTKLEL
K
or comprising the sequence
(SEQ ID NO: 39)
QVQLVQSGAEVVKPGASVKISCKASGYTFTDHAIHWVKQNPGQRLEWIG
YFSPGNDDFKYNERFKGKATLTADTSASTAYVELSSLRSEDTAVYFCTR
SLNMAYWGQGTLVTVSSGSTS
and the sequence
(SEQ ID NO: 40)
SSDIVMSQSPDSLAVSLGERVTLNCKSSQSLLYSGNQKNYLAWYQQKPG
QSPKLLIYWASARESGVPDRFSGSGSGTDFTLTISSVQAEDVAVYYCQQ
YYSYPLTFGAGTKLELK;
joined by a flexible linker
(SEQ ID NO: 33)
QVQLVQSGAEVVKPGASVKISCKASGYTFTDHAIHWVKQNPGQRLEWIG
YFSPGNDDFKYSQKFQGKATLTADTSASTAYVELSSLRSEDTAVYFCTR
SLNMAYWGQGTLVTVSSGSTSGGGSGGGSGGGGSSDIVMSQSPDSLAVS
LGERVTLNCKSSQSVLYSSNSKNYLAWYQQKPGQSPKLLIYWASTRESG
VPDRFSGSGSGTDFTLTISSVQAEDVAVYYCQQYYSYPLSFGAGTKLEL
K;
or
(SEQ ID NO: 34)
QVQLVQSGAEVKKPGASVKVSCKASGYTFTDHAIHWVRQAPGQRLEWMG
YFSPGNDDFKYSQKFQGRVTITADTSASTAYMELSSLRSEDTAVYFCTR
SLNMAYWGQGTLVTVSSGSTSGGGSGGGSGGGGSSDIVMTQSPDSLAVS
LGERATINCKSSQSLLYSSNSKNYLAWYQQKPGQPPKLLIYWASTRESG
VPDRFSGSGSGTDFTLTISSLQAEDVAVYYCQQPYSYPLSFGAGTKLEL
K.

A useful TAG72 CAR can consist of or comprises the amino acid sequence of SEQ ID NO: 35, 36, or 37 (mature CAR lacking a signal sequence) or the TAG72 CAR can consist of or comprise the amino acid sequence of SEQ ID NO:29, 31, or 32 (immature CAR having a GMCSFRa signal sequence). The CAR and can be expressed in a form that includes a signal sequence, e.g., a human GM-CSF receptor alpha signal sequence (MLLLVTSLLLCELPHPAFLLIP; SEQ ID NO:41). The CAR can be expressed with additional sequences that are useful for monitoring expression, for example, a T2A skip sequence and a truncated EGFRt. Thus, the CAR can comprise or consist of the amino acid sequence of SEQ ID Nos: 29, 31, or 32 or can comprise or consist of an amino acid sequence that is at least 95%, 96%, 97%, 98% or 99% identical to SEQ ID Nos: 29, 31, or 32. The CAR can comprise or consist of the amino acid sequence of any of SEQ ID Nos: 29, 31, or 32 with up to 1, 2, 3, 4 or 5 amino acid changes (preferably conservative amino acid changes).

### Spacer Region

The CAR described herein can include a spacer located between the TAG72 targeting domain (i.e., a TAG72 targeted ScFv or variant thereof) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG (SEQ ID NO: 2) |
| IgG4 hinge (S→P) | 12 aa | ESKYGPPCPPCP (SEQ ID NO: 3) |
| (S228P) | | |
| IgG4 hinge | 12 aa | ESKYGPPCPSCP (SEQ ID NO: 4) |
| IgG4 hinge (S228P) + linker | 22 aa | ESKYGPPCPPCPGGGSSGGGSG (SEQ ID NO: 5) |
| CD28 hinge | 39 aa | IEVMYPPPYLDNEKSNGTIIHVKGKHLCPSPLFPGPSKP (SEQ ID NO: 6) |
| CD8 hinge-48aa | 48 aa | AKPTTTPAPRPPTPAPTIASQPLSLRPEACRPAAGGAVHTRGLDFACD (SEQ |
| | | ID NO: 7) |
| CD8 hinge-45aa | 45 aa | TTTPAPRPPTPAPTIASQPLSLRPEACRPAAGGAVHTRGLDFACD (SEQ ID |
| | | NO: 8) |
| IgG4(HL-CH3) | 129 aa | ESKYGPPCPPCPGGGSSGGGSGGQPREPQVYTLPPSQEEMTKNQVSLTCL |
| (includes S228P in hinge) | | VKGFYPSDIAVEWESNGQPENNYKTTPPVLDSDGSFFLYSRLTVDKSRWQE |
| | | GNVFSCSVMHEALHNHYTQKSLSLSLGK (SEQ ID NO: 9) |
| IgG4(L235E,N297Q) | 229 aa | ESKYGPPCPSCPAPEFEGGPSVFLFPPKPKDTLMISRTPEVTCVVVDVSQED |
| | | PEVQFNWYVDGVEVHQAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 10) |
| IgG4(S228P, L235E, N297Q) | 229 aa | ESKYGPPCPPCPAPEFEGGPSVFLFPPKPKDTLMISRTPEVTCVVVDVSQED |
| | | PEVQFNWYVDGVEVHQAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 11) |
| IgG4(CH3) | 107 aa | GQPREPQVYTLPPSQEEMTKNQVSLTCLVKGFYPSDIAVEWESNGQPENN |
| | | YKTTPPVLDSDGSFFLYSRLTVDKSRWQEGNVFSCSVMHEALHNHYTQKSL |
| | | SLSLGK (SEQ ID NO: 12) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise a IgG4 hinge region having the sequence ESKYGPPCPSCP (SEQ ID NO:4) or ESKYGPPCPPCP (SEQ ID NO:3). The hinge/linger region can also comprise the sequence ESKYGPPCPPCP (SEQ ID NO:3) followed by the linker sequence GGGSSGGGSG (SEQ ID NO:2) followed by IgG4 CH3 sequence GQPREPQVYTLPPSQEEMTKNQVSLTCLVKGFYPSDIAVEWESNGQPENNYKTTPPVLD SDGSFFLYSRLTVDKSRWQEGNVFSCSVMHEALHNHYTQKSLSLSLGK (SEQ ID NO:12). Thus, the entire linker/spacer region can comprise the sequence: ESKYGPPCPPCPGGGSSGGGSGGQPREPQVYTLPPSQEEMTKNQVSLTCLVKGFYPSDIA VEWESNGQPENNYKTTPPVLDSDGSFFLYSRLTVDKSRWQEGNVFSCSVMHEALHNHY TQKSLSLSLGK (SEQ ID NO:11). In some cases, the spacer has 1, 2, 3, 4, or 5 single amino acid changes (e.g., conservative changes) compared to SEQ ID NO:11. In some cases, the IgG4 Fc hinge/linker region that is mutated at two positions (L235E; N297Q) in a manner that reduces binding by Fc receptors (FcRs).

### Transmembrane Domain

A variety of transmembrane domains can be used in the. Table 2 includes examples of suitable transmembrane domains. Where a spacer region is present, the transmembrane domain is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL |
| | | | (SEQ ID NO: 13) |
| CD28 | NM_006139 | 27 aa | FWVLVVVGGVLACYSLLVTVA |
| | | | FIIFWV |
| | | | (SEQ ID NO: 14) |
| CD28(M) | NM_006139 | 28 aa | MFWVLVVVGGVLACYSLLVTV |
| | | | AFIIFWV |
| | | | (SEQ ID NO: 15) |
| CD4 | M35160 | 22 aa | MALIVLGGVAGLLLFIGLGIF |
| | | | F (SEQ ID NO: 16) |
| CD8tm | NM_001768 | 21 aa | IYIWAPLAGTCGVLLLSLVIT |
| | | | (SEQ ID NO: 17) |
| CD8tm2 | NM_001768 | 23 aa | IYIWAPLAGTCGVLLLSLVIT |
| | | | LY (SEQ ID NO: 18) |
| CD8tm3 | NM_001768 | 24 aa | IYIWAPLAGTCGVLLLSLVIT |
| | | | LYC (SEQ ID NO: 19) |
| 41BB | NM_001561 | 27 aa | IISFFLALTSTALLFLLFFLT |
| | | | LRFSVV |
| | | | (SEQ ID NO: 20) |

### Costimulatory Domain

The costimulatory domain can be any domain that is suitable for use with a CD3ζ signaling domain. In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:24). In some cases, the 4-1BB co-signaling domain has 1, 2, 3, 4 of 5 amino acid changes (preferably conservative) compared to SEQ ID NO:24.

The costimulatory domain(s) are located between the transmembrane domain and the CD3ζ signaling domain. Table 3 includes examples of suitable costimulatory domains together with the sequence of the CD3ζ signaling domain.

TABLE 3
CD3ζ Domain and Examples of
Costimulatory Domains

| Name | Accession | Length | Sequence |
|---|---|---|---|
| CD3ζ | J04132.1 | 113 aa | RVKFSRSADAPAYQQGQNQLYN |
| | | | ELNLGRREEYDVLDKRRGRDPE |
| | | | MGGKPRRKNPQEGLYNELQKDK |
| | | | MAEAYSEIGMKGERRRGKGHDG |
| | | | LYQGLSTATKDTYDALHMQALP |
| | | | PR (SEQ ID NO: 21) |
| CD28 | NM_006139 | 42 aa | RSKRSRLLHSDYMNMTPRRPGP |
| | | | TRKHYQPYAPPRDFAAYRS |
| | | | (SEQ ID NO: 22) |
| CD28gg* | NM_006139 | 42 aa | RSKRSRGGHSDYMNMTPRRPGP |
| | | | TRKHYQPYAPPRDFAAYRS |
| | | | (SEQ ID NO: 23) |
| 41BB | NM_001561 | 42 aa | KRGRKKLLYIFKQPFMRPVQTT |
| | | | QEEDGCSCRFPEEEEGGCEL |
| | | | (SEQ ID NO: 24) |
| OX40 | | 42 aa | ALYLLRRDQRLPPDAHKPPGGG |
| | | | SFRTPIQEEQADAHSTLAKI |
| | | | (SEQ ID NO:25) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and a short linker consisting of 2-10, e.g., 3 amino acids (e.g., GGG) is can be positioned between the costimulatory domain and the CD3ζ signaling domain.

### CD3ζ Signaling Domain

The CD3ζ Signaling domain can be any domain that is suitable for use with a CD3ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO:21). In some cases, the CD3ζ signaling has 1, 2, 3, 4 of 5 amino acid changes (preferably conservative) compared to SEQ ID NO:21.

### Truncated EGFR

The CD3ζ signaling domain can be followed by a ribosomal skip sequence (e.g., LEGGGEGRGSLLTCGDVEENPGPR; SEQ ID NO:27) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHILPVAFR GDSFTHTPPLDPQELDILKTVKEITGFLLIQAWPENRTDLHAFENLEIIRGRTKQHGQFSL AVVSLNITSLGLRSLKEISDGDVIISGNKNLCYANTINWKKLFGTSGQKTKIISNRGENSC KATGQVCHALCSPEGCWGPEPRDCVSCRNVSRGRECVDKCNLLEGEPREFVENSECIQC HPECLPQAMNITCTGRGPDNCIQCAHYIDGPHCVKTCPAGVMGENNTLVWKYADAGH VCHLCHPNCTYGCTGPGLEGCPTNGPKIPSIATGMVGALLLLLVVALGIGLFM (SEQ ID NO:28). In some cases, the truncated EGFR has 1, 2, 3, 4 of 5 amino acid changes (preferably conservative) compared to SEQ ID NO:28.

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In some cases, the TAG72 CAR can be produced using a vector in which the CAR open reading frame is followed by a T2A ribosome skip sequence and a truncated EGFR (EGFRt), which lacks the cytoplasmic signaling tail. In this arrangement, co-expression of EGFRt provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt incorporated in the TAG72 CAR lentiviral vector can act as suicide gene to ablate the CAR+ T cells in cases of treatment-related toxicity.

The CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte cell line, and most preferably an autologous T lymphocyte cell line.

Various T cell subsets isolated from the patient can be transduced with a vector for CAR expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of an TAG72 CAR as well as a non-immunogenic surface marker for in vivo detection, ablation, and potential ex vivo selection. The activated/genetically modified TAG72 central memory T cells can be expanded in vitro with IL-2/IL-15 and then cryopreserved.

### DESCRIPTION OF DRAWINGS

FIG. 1A-1D shows cartoon representation of TAG72-BBζ CAR T cells and shows results of TAG72-BBζ CAR T cells cultured with purified TAG72. (A) Diagram of the lentiviral expression cassette with TAG72-CARs containing the humanized scFv (CC49 clone) targeting TAG72, with a 129 amino acid modified human IgG4 Fc linker (void of the CH2 domain, ACH2), a CD4 transmembrane domain, a cytoplasmic 4-1BB costimulatory domain, and a cytolytic CD3ζ domain. A truncated non-signaling CD19 (CD19t), separated from the CAR sequence by a T2A ribosomal skip sequence, was expressed for identifying lentivirally transduced T cells. (B) Mock (untransduced) and TAG72-BBζ CAR T cells were evaluated by flow cytometry for CD19t expression to detect lentiviral transduction of CARs (left) or Protein L to detect the scFv (right). (C) CD4 and CD8 expression in Mock (top) and TAG72− BBζ CAR T cells (bottom). (D) Activation (expression of CD137) was assessed by flow cytometry with in vitro stimulated CAR T cells against soluble or plate-bound purified TAG72 antigen for 24 h at indicated protein amounts (units).

FIG. 2A-2B shows results of TAG72-BBζ CAR T cell activation against purified TAG72 antigen. (A) Activation (expression of CD69) was assessed by flow cytometry with in vitro stimulated CAR T cells against soluble or plate-bound purified TAG72 antigen for 24 h at indicated protein amounts (units). (B) IFNγ production by ELISA from TAG72-BBζ CAR T cells against plate-bound purified TAG72 antigen.

FIG. 3A-3H shows results from experiments with TAG72-BBζ CAR T cells cultured with TAG72-positive and TAG72-negative cancer cells. (a) Flow cytometric analysis of TAG72 surface expression on multiple ovarian and colorectal (LS174T) cancer cell lines. (b) Quantification of tumor killing by TAG72-BBζ CAR T cells relative to Mock following a 24 and 72 h co-culture with antigen-positive and -negative tumor targets as described in Materials and Methods. (c) TAG72-BBζ CAR T cell expansion at 24 and 72 h following co-culture with indicated tumor targets. (d,e) IFNγ and IL-2 levels in supernatant quantified by ELISA from Mock or TAG72-BBζ CAR T cells following a 24 and 72 h co-culture with indicated tumor targets. (F) Flow cytometric analysis of TAG72 surface expression on primary human ovarian cancer cells harvested from patient ascites (OAS) after 72 h in culture. (G) Quantification of tumor killing and (H) IFNγ production by TAG72-BBζ CAR T cells relative to Mock following a 72 h co-culture with freshly thawed OAS cells.

FIG. 4 shows results of flow cytometric analysis of TAG72 expression on ascites from OVCAR3 or OV90 tumor-bearing mice.

FIG. 5 shows results TAG72-BBζ CAR T cell-mediated tumor killing of OVCAR3 cells in the presence or absence of 10 units of soluble TAG72 in a 24 h co-culture assay.

FIG. 6A-6F shows results from experiments with regional intraperitoneal delivery of TAG72-BBζ CAR T cells in of OVCAR3 tumor-bearing mice. (A) Schematic illustrating i.p. engraftment of 5.0×106 OVCAR3(eGFP/ffluc) tumor cells in NSG mice, followed by either i.v. or i.p. delivery of 5.0×106 Mock or TAG72-BBζ CAR T cells on day 14 post tumor injection. (B) Representative bioluminescent flux imaging of mice treated i.v. or i.p. with Mock or TAG72-BBζ CAR T cells. (C) Quantification of flux (each mouse) from OVCAR3(eGFP/ffluc) tumor-bearing mice treated i.v. or i.p. with Mock or TAG72-BBζ CAR T cells. N=3 per group. (D) Kaplan-Meier survival for Mock and TAG72-BBζ CAR T cell treated mice. N≥4 mice per group. Data are representative of or combined from two independent experiments. (E) Quantification of TAG72-BBζ CAR T cells per uL blood at 6, 13, and 29 days post treatment. N=4 per group. (F) Representative flow cytometric analysis of the frequency of human CD45+ (hCD45) and mouse CD45+ (mCD45) cells in the i.p. cavity of tumor-bearing mice at day 6 and 13 post treatment. Representative images from two independent experiments.

FIG. 7 shows quantification of human CD45+ cells in OVCAR3 model; quantification of human CD45+ cells per uL blood at 6, 13, and 29 days post treatment. N=4 per group.

FIG. 8A-8B shows results of TAG72-BBζ CAR T cells anti-tumor activity in OV90 tumor-bearing mice in vivo delivered either by i.p. or by i.v.; (A) Quantification of flux (each mouse) from OV90(eGFP/ffluc) tumor-bearing mice treated i.v. or i.p. with Mock or TAG72-BBζ CAR T cells. (B) Kaplan-Meier survival for Mock and TAG72-BBζ CAR T cell treated mice. N≥4 mice per group.

FIG. 9A-9F show results of experiments with either single or repeat regional administration of TAG72-BBζ CAR T cells in OV90 tumor-bearing mice. (A) Schematic illustrating i.p. engraftment of 5.0×106 OV90(eGFP/ffluc) tumor cells in NSG mice, followed by either single or repeat i.p. treatment with 5.0×106 Mock or TAG72-BBζ CAR T cells on day 8 post tumor infection. (B) Representative bioluminescent flux imaging of mice treated i.p. with a single or repeat treatment of Mock or TAG72-BBζ CAR T cells. (C) Quantification of flux (each mouse) from OV90(eGFP/ffluc) tumor-bearing mice with single or repeat i.p. treatment of Mock or TAG72− BBζ CAR T cells. (D) Analysis of relative tumor growth kinetics at start of treatment (top) and at peak therapy (bottom) time points for all mice. Mann-Whitney test was performed to calculate p values. (E) Kaplan-Meier survival for Mock and TAG72-BBζ CAR T cell treated mice. N≥5 mice per group. (F) Histology of human CD3 cells in tumors harvested from single and repeat treated mice at days 42 and 70 post tumor injection (top: 10× magnification, bottom: 40× magnification). Data are representative of two independent experiments.

FIG. 10A-10B shows quantification of human CD45+ cells in OV90 model; quantification of human CD45+ cells per uL blood at 7, 14, and 34 days post treatment. N=4 per group.

FIG. 11A-11E show results of tumor-associated glycoprotein antigen heterogeneity in ovarian cancer and experiments quantifying CAR T cell-mediated antigen escape. (A) Flow cytometric analysis of TAG72, MUC16, and MUC1 surface expression on OVCAR8, OVCAR3, and OV90 human ovarian cancer cell lines. (B) Histology of TAG72, MUC16, and MUC1 expression in i.p. solid tumors harvested from Mock and TAG72-BBζ CAR T cell treated OVCAR3 tumor-bearing mice at day 99 post treatment. 10× magnification. (C) Histology of TAG72 expression on solid tumors harvested from single and repeat treated OV90 tumor-bearing mice at day 42, 70, and 105 post tumor injection. 10× magnification. (D) Flow cytometric analysis of TAG72 expression in OV90 tumor cells harvested from ascites at indicated time points from mice that received single or repeat i.p. treatment. (E) TAG72 expression on OVCAR3 cells at day 4 following co-culture with Mock or TAG72-BBζ CAR T cells (1:10 E:T ratio), and on tumor cells that grew out at day 28.

FIG. 12 shows the annotated polypeptide sequence of hTag72scFv-IgG4(HL-CH3)-CD4tm-41BB-Zeta-T2A-CD19t (SEQ ID NO:26 with the T2A and CD19t; SEQ ID NO:29 without the T2A and CD19t). SEQ ID NO:35 without the GMCSFRa signal peptide, T2A and CD19t.

FIGS. 13A-13C show tumor killing, activation, and T cell proliferation of humanized TAG72 CAR T cells. (A) OV90 and OVCAR3 cells were co-cultured for 72 hours with either Mock, IDEC, V15 or V59/15 variant TAG72 CAR T cells at an E:T of 1:2. Tumor killing is represented as % killing relative to mock-treated conditions. (B) T cell activation was analyzed from 72 hour co-culture assays by flow cytometry staining of surface CD137 expression. (C) T cell proliferation (fold expansion) at 72 hours was determined relative to T cell counts plated on day 0.

FIGS. 14A-14B show results of experiments with either single or repeat regional administration of humanized TAG72 CAR T cells in OV90 tumor-bearing mice. (A) Endogenous expression of TAG72 antigen on OV90 tumor cell line was determined by flow cytometry. OV90-ffluc cells were injected into the intraperitoneal (i.p.) cavity of NSG mice and tracked by bioluminescent imaging and reported as flux (photos/sec). At 8 days post tumor injection, either a single or repeat dose of 5.0×10⁶ Mock, IDEC, or V15 variants of TAG72 CAR T cells administered regionally into the i.p. cavity of tumor-bearing mice. (B) Tumor burden of single or repeat T cell-treated mice was quantified by bioluminescent imaging. Dashed vertical lines indicate time points of initial and repeated treatment with T cells.

FIGS. 15A-15C show results of i.v. administered humanized TAG72 CAR T cells in OVCAR3 tumor-bearing mice. (A) Endogenous surface TAG72 expression was analyzed by flow cytometry on OVCAR3 tumor cells. OVCAR3-ffluc tumors were then injected into the i.p. cavity of NSG mice, and treated i.v. with a single dose of 5.0×10⁶ Mock, IDEC, or V15 variant TAG72 CAR T cells. (B) Tumor burden of single dose treated mice was quantified by bioluminescent imaging and reported as flux (photos/sec). Dashed vertical lines indicate time point of treatment with T cells. (C) Quantification of either Mock, or IDEC and V15 TAG72 CAR T cell persistence and proliferation was quantified in the blood by flow cytometry (CAR+ cells per uL of blood) in mice at day 6, 13, and 29 post T cell treatment, and highlight increased persistence and proliferation of V15 variant TAG72 CAR T cells over IDEC.

FIGS. 16A-16E show varying humanized V15-CAR design impacts in vitro antitumor T cell functional activity. (A) CAR expression stability of seven TAG72-CAR T cells variants (with the V15 scFv clone). (B-E) In vitro tumor killing activity, T cell proliferation, CD137+ activation indicator, and PD-1+ exhaustion indicator (72 hours), of CAR T cells against TAG72-negative (DU145, OVCAR8), and TAG72-positive (OVCAR3, OV90, and OVCAR8-sTn) expressing tumor cells.

FIG. 17 shows varying humanized V15-CAR design impacts in vitro cytokine production of TAG72-CAR T cells. In vitro IFNγ production (24 hours), of CAR T cells against TAG72-negative (DU145, OVCAR8), and TAG72-positive (OVCAR3, OV90, OVCAR8-sTn) expressing tumor cells.

FIGS. 18A-18B show real-time long-term killing and proliferation of humanized TAG72 CAR T cells. (A) Real time cytotoxicity assay was performed using xCelligence technology with OV90 cells. The four T cells populations were plated at an effector to target ratio of 1-to-20 and observed for 10 days. Cell Index is indicative of live tumor count. (B) At endpoint, remaining cells were collected and analyzed by flow cytometry.

FIG. 19 shows the annotated polypeptide sequence of Tag72scFv(IDEC)-IgG4(HL-CH3)-CD4tm-41BB-Zeta without the with the T2A and CD19t (SEQ ID NO:30). SEQ ID NO:35 without the GMCSFRa signal peptide.

FIG. 20 shows the annotated polypeptide sequence of Tag72scFv(v15)-IgG4(HL-CH3)-CD4tm-41BB-Zeta without the with the T2A and CD19t (SEQ ID NO:31). SEQ ID NO:36 without the GMCSFRa signal peptide.

FIG. 21 shows the annotated polypeptide sequence of Tag72scFv(v59_v15)-IgG4(HL-CH3)-CD4tm-41BB-Zeta without the with the T2A and CD19t (SEQ ID NO:32). SEQ ID NO:37 without the GMCSFRa signal peptide.

### DETAILED DESCRIPTION

In this disclosure the generation and anti-tumor efficacy of a second-generation CAR T cell with a humanized anti-human TAG72 scFv antigen-binding domain and a 4-1BB intracellular co-stimulatory signaling domain (TAG72-BBζ) are described. The TAG72-BBζ CAR T cells exhibited potent antigen-dependent cytotoxicity against multiple TAG72-expressing human ovarian cancer cell lines and epithelial cells derived from patient ovarian cancer ascites grown in cell culture. Regional intraperitoneal in vivo delivery of TAG72-BBζ CAR T cells in peritoneal ovarian tumor models conferred elimination of antigen-positive disease and extension of mice overall survival. In contrast, intravenous CAR T cell delivery was ineffective in controlling disease. Additionally, repeat regional infusions of TAG72-BBζ CAR T cells promoted more durable control of disease compared to single treatment. These preclinical findings support TAG72-BBζ CAR T cells as a viable therapeutic option for ovarian cancers, and also highlight its broader application for multiple TAG72-expressing solid cancers.

### Examples

The invention is further described in the following examples, which do not limit the scope of the invention described in the claims.

### Materials and Methods

The following materials and methods were used in the Examples set forth herein.

### Cell Lines

The epithelial ovarian cancer line OVCAR3 (ATCC HTB-161) was cultured in RPMI-1640 (Lonza) containing 20% fetal bovine serum (FBS, Hyclone) and 1× antibiotic-antimycotic (1×AA, Gibco) (complete RPMI). The epithelial ovarian cancer line derived from metastatic ascites OV90 (CRL-11732) was cultured in a 1:1 mixture of MCDB 105 medium (Sigma) and Medium 199 (Thermo) adjusted to pH of 7.0 with sodium hydroxide (Sigma) and final 20% FBS and 1×AA. The epithelial-endometroid ovarian cancer line COV362.4 (Sigma) was cultured in Dulbecco's Modified Eagles Medium (DMEM, Life Technologies) containing 10% FBS, 1×AA, 25 mM HEPES (Irvine Scientific), and 2 mM L-Glutamine (Fisher Scientific) (complete DMEM). The epithelial ovarian cancer line OVCAR8 was a generous gift from Dr. Carlotta Glackin at City of Hope and was cultured in complete RPMI-1640. The epithelial ovarian cancer line SKOV3 (ATCC HTB-77) and the colon epithelial cancer line LS174T (ATCC CL-188) were cultured in complete DMEM. All cells were cultured at 37° C. with 5% CO2.

### DNA Constructs and Lentivirus Production

Tumor cells were engineered to express enhanced green fluorescent protein and firefly luciferase (eGFP/ffluc) by transduction with epHIV7 lentivirus carrying the eGFP/ffluc fusion under the control of the EF1α promoter as described previously (22). The humanized scFv sequence used in the CAR construct was obtained from a monoclonal antibody clone huCC49 that targets TAG72 (17). The extracellular spacer domain included the 129-amino acid middle-length CH2-deleted version (ACH2) of the IgG4 Fc spacer (23). The intracellular co-stimulatory signaling domain contained was a 4-1BB with a CD4 transmembrane domain. The CD3ζ cytolytic domain was previously described (22). The CAR sequence was separated from a truncated CD19 gene (CD19t) by a T2A ribosomal skip sequence, and cloned in an epHIV7 lentiviral backbone under the control of the EF1α promoter.

Lentivirus was generated as previously described (22, 24). Briefly, 293T cells were transfected with packaging plasmid and CAR lentiviral backbone plasmid using a modified calcium phosphate method. Viral supernatants were collected after 3 to 4 days and treated with 2 mM magnesium and 25 U/mL Benzonase® endonuclease (EMD Millipore). Supernatants were concentrated via high-speed centrifugation and lentiviral pellets were resuspended in phosphate-buffered saline (PBS)-lactose solution (4 g lactose per 100 mL PBS), aliquoted and stored at −80° C. Lentiviral titers were quantified using HT1080 cells based on CD19t expression.

### T Cell Isolation, Lentiviral Transduction, and Ex Vivo Expansion

Leukapheresis products were obtained from consented research participants (healthy donors) under protocols approved by the City of Hope Internal Review Board (IRB). On the day of leukapheresis, peripheral blood mononuclear cells (PBMC) were isolated by density gradient centrifugation over Ficoll-Paque (GE Healthcare) followed by multiple washes in PBS/EDTA (Miltenyi Biotec). Cells were rested overnight at room temperature (RT) on a rotator, and subsequently washed and resuspended in X-VIVO T cell medium (Lonza) containing 10% FBS (complete X-VIVO). Up to 5.0×109 PBMC were incubated with anti-CD14 and anti-CD25 microbeads (Miltenyi Biotec) for 30 min at RT and magnetically depleted using the CliniMACS® system (Miltenyi Biotec) according to the manufacturer's protocol and these were termed depleted PBMCs (dPBMC). dPBMC were frozen in CryoStor® CS5 (StemCell Technologies) until further processing.

T cell activation and transduction was performed as described previously (22). Briefly, freshly thawed dPBMC were washed once and cultured in complete X-VIVO containing 100 U/mL recombinant human IL-2 (rhIL-2, Novartis Oncology) and 0.5 ng/mL recombinant human IL-15 (rhIL-15, CellGenix). For CAR lentiviral transduction, T cells were cultured with CD3/CD28 Dynabeads® (Life Technologies), protamine sulfate (APP Pharmaceuticals), cytokine mixture (as stated above) and desired lentivirus at a multiplicity or infection (MOI) of 1 the day following bead stimulation. Cells were then cultured in and replenished with fresh complete X-VIVO containing cytokines every 2-3 days. After 7 days, beads were magnetically removed, and cells were further expanded in complete X-VIVO containing cytokines to achieve desired cell yield. CAR T cells were positively selected for CD19t using the EasySep™ CD19 Positive Enrichment Kit I or II (StemCell Technologies) according to the manufacturer's protocol. Following further expansion, cells were frozen in CryoStor® CS5 prior to in vitro functional assays and in vivo tumor models. Purity and phenotype of CAR T cells were verified by flow cytometry.

### Flow Cytometry

For flow cytometric analysis, cells were resuspended in FACS buffer (Hank's balanced salt solution without Ca2+, Mg2+, or phenol red (HBSS−/−, Life Technologies) containing 2% FBS and 1×AA). Cells were incubated with primary antibodies for 30 minutes at 4° C. in the dark. For secondary staining, cells were washed twice prior to 30 min incubation at 4° C. in the dark with either Brilliant Violet 510 (BV510), fluorescein isothiocyanate (FITC), phycoerythrin (PE), peridinin chlorophyll protein complex (PerCP), PerCP-Cy5.5, PE-Cy7, allophycocyanin (APC), or APC-Cy7 (or APC-eFluor780)-conjugated antibodies. Antibodies against CD3 ζ BD Biosciences, Clone: SK7), CD4 (BD Biosciences, Clone: SK3), CD8 (BD Biosciences, Clone: SKi), CD14 (BD Biosciences, Clone: M(DP9), CD19 (BD Biosciences, Clone: SJ25C1), CD25 (BD Biosciences, Clone: 2A3), mouse CD45 (BioLegend, Clone: 30-F11), CD45 (BD Biosciences, Clone: 2D1), CD69 (BD Biosciences, Clone: L78), CD137 (BD Biosciences, Clone: 4B4-1), MUC1 (BioLegend, Clone 16A), MUC16 (Abcam, Clone X75 or EPSISR23), biotinylated Protein-L (GenScript USA) (25), TAG72 (Clone, muCC49), Donkey Anti-Rabbit Ig (Invitrogen), Goat Anti-Mouse Ig (BD Biosciences), and streptavidin (BD Biosciences) were used. Cell viability was determined using 4′, 6-diamidino-2-phenylindole (DAPI, Sigma). Flow cytometry was performed on a MACSQuant Analyzer 10 (Miltenyi Biotec), and the data was analyzed with FlowJo software (v10, TreeStar).

### In Vitro Tumor Killing and T Cell Functional Assays

For tumor killing assays, CAR T cells and tumor targets were co-cultured at indicated effector:tumor (E:T) ratios in complete X-VIVO in the absence of exogenous cytokines in 96-well plates for 24 to 72 h and analyzed by flow cytometry as described above. Tumor killing by CAR T cells was calculated by comparing CD45-negative cell counts relative to that observed when targets were co-cultured with Mock (untransduced) T cells. For T cell activation assays, CAR T cells and tumor targets were co-cultured at the indicated E:T ratios in complete X-VIVO in the absence of exogenous cytokines in 96-well plates for the indicated time points and analyzed by flow cytometry for specific markers of T cell activation. Frozen, uncultured patient primary ovarian cancer ascites (OAS3, OAS4, and OAS7) were thawed, directly analyzed for TAG72 expression, and evaluated in T cell functional assays. Briefly, ascites fluid from ovarian cancer patients was obtained from City of Hope National Medical Center (COH) surgical staff in a sterile vacuum container with approval from the COH Institutional Review Board (IRB) and Office of Human Subjects Protection. The COH IRB waived the need for written informed consent as all samples were de-identified and ascites was discard material as previously described (26).

For T cell activation assays on plate-bound antigen, purified soluble TAG72 antigen (BioRad) was plated in duplicate at indicated TAG72 units overnight at 4° C. in 1×PBS in 96-well flat bottom high-affinity plates (Corning). A total of 104 TAG72-BBζ CAR T cells were then added in a fixed volume of 100p L to each well and incubated for indicated times prior to collection of cells for analysis of activation markers (CD69, CD137) by flow cytometry. Supernatants were also collected for analysis of cytokine production.

### ELISA Cytokine Assays

Supernatants from tumor killing assays or CAR T cell activation assays on plate-bound TAG72 antigen were collected at indicated times and frozen at −20° C. for further use. Supernatants were then analyzed for secreted human IFNγ and IL-2 according to the Human IFNγ and IL-2 ELISA Ready-SET-GO!® ELISA kit manufacturer's protocol, respectively. Plates were read at 450 nm using a Wallac Victor3 1420 Counter (Perkin-Elmer) and the Wallac 1420 Workstation software.

### In Vivo Tumor Studies

All animal experiments were performed under protocols approved by the City of Hope Institutional Animal Care and Use Committee. For in vivo tumor studies, OVCAR3 and OV90 cells (5.0×106) were prepared in a final volume of 500 μl HBSS−/− and engrafted in 6 to 8 week old female NSG mice by intraperitoneal (i.p.) injection. Tumor growth was monitored at least once a week via biophotonic imaging (Xenogen, LagoX) and flux signals were analyzed with Living Image software (Xenogen). For imaging, mice were i.p. injected with 150 μL D-luciferin potassium salt (Perkin Elmer) suspended in PBS at 4.29 mg/mouse. Once flux signals reached desired levels, day 8 for OV90 and day 14 for OVCAR3, T cells were prepared in 1×PBS, and mice were treated with 500 μL i.p. or 200 μL intravenous (i.v.) injection of 5.0×106 Mock or TAG72-BBζ CAR T cells. In the OV90 tumor model, we tested the impact of repeat treatment with i.p. TAG72-BBζ CAR T cells starting at day 8, followed by treatments at additional indicated days post tumor engraftment. Humane endpoints were used in determining survival. Mice were euthanized upon signs of distress such as a distended belly due to ascites, labored or difficulty breathing, apparent weight loss, impaired mobility, or evidence of being moribund. At pre-determined time points or at moribund status, mice were euthanized and tissues and/or ascites fluid were harvested and processed for flow cytometry and immunohistochemistry as described below.

Peripheral blood was collected from isoflurane-anesthetized mice by retro-orbital (RO) bleed through heparinized capillary tubes (Chase Scientific) into polystyrene tubes containing a heparin/PBS solution (1000 units/mL, Sagent Pharmaceuticals). Volume of each RO blood draw (approximately 120 μL/mouse) was recorded for cell quantification per μL blood. Red blood cells (RBCs) were lysed with 1× Red Cell Lysis Buffer (Sigma) according to the manufacturer's protocol and then washed, stained, and analyzed by flow cytometry as described above. Cells from i.p. ascites fluid was collected from euthanized mice by injecting 5 mL cold 1×PBS into the i.p. cavity, which was drawn up via syringe and stored on ice until further processing. RBC-depleted ascites was washed, stained, and analyzed by flow cytometry for tumor-associated glycoprotein expression and CAR T cells using antibodies and methods described above.

### Immunohistochemistry

Tumor tissue was fixed for up to 3 days in 4% paraformaldehyde (4% PFA, Boston BioProducts) and stored in 70% ethanol until further processing. Immunohistochemistry was performed by the Pathology Core at City of Hope. Briefly, paraffin-embedded sections (10 μm) were stained with hematoxylin & eosin (H&E, Sigma-Aldrich), mouse anti-human CD3 (DAKO), mouse anti-human TAG72 (AB16838, Abcam), rabbit anti-human MUC1 (AB45167, Abcam), MUC16 (AB1107, Abcam). Images were obtained using the Nanozoomer 2.0HT digital slide scanner and the associated NDP.view2 software (Hamamatzu).

### Statistical Analysis

Data are presented as mean±SEM, unless otherwise stated. Statistical comparisons between groups were performed using the unpaired two-tailed Student's t test to calculate p value, unless otherwise stated. *p<0.05, **p<0.01, ***p<0.001; NS, not significant.

### Example 1: Construction of TAG72-CAR T Cells Containing a 4-1BB Intracellular Co-Stimulatory Domain and Validation that TAG72-BBζ CAR T Cells Exhibit Activity Against TAG72

To determine if TAG72-CAR T cells containing a 4-1BB intracellular co-stimulatory domain effectively demonstrate activation against purified TAG72, the aforementioned cells were grown in presence of increasing amounts of either soluble TAG72 or plate-bound TAG72 and CD137 expression, an indicator of activation, was measured.

### Results

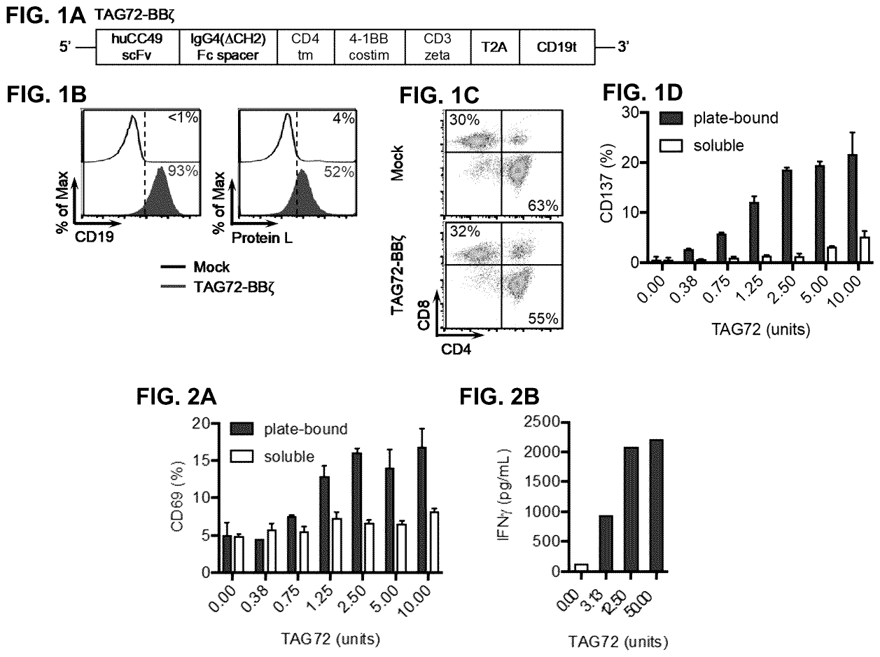

TAG72-BBζ CAR lentivirus was used to transduce human healthy donor-derived peripheral blood mononuclear cells depleted of CD14+ and CD25+ cells (dPBMC), as previously described (Priceman S J, Gerdts E A, Tilakawardane D, Kennewick K T, Murad J P, Park A K, Jeang B, Yamaguchi Y, Yang X, Urak R, Weng L, Chang W C, Wright S, Pal S, Reiter R E, Wu A M, Brown C E, Forman S J. Co-stimulatory signaling determines tumor antigen sensitivity and persistence of CAR T cells targeting PSCA+ metastatic prostate cancer. Oncoimmunology. 2018; 7(2):e1380764). TAG72-BBζ CAR T cells were enriched during the manufacturing process (based on CD19t+ selection) and were stably expressed on the surface of T cells (FIG. 1B). CAR T cells expanded ex vivo with similar kinetics and comparable CD4:CD8 ratios to Mock (untransduced) T cells (FIG. 1C). Importantly, and as a first measure of CAR T cell activation against TAG72, TAG72-BBζ CAR T cells exhibited dose-dependent CD137 expression on the surface when cultured with plate-bound, but not soluble, purified TAG72 (FIG. 1D). Additionally, TAG72-BBζ CAR T cells exhibited dose-dependent induction of other activator indicators, specifically cell-surface CD69 expression and IFNγ release, when cultured with plate-bound TAG72, but not soluble, purified TAG72 (FIG. 2).

### Example 2: Validation that TAG72-BBζ CAR T Cells Selectively Target and Exhibit Activation Against TAG72-Positive Ovarian Cancer Cells In Vitro

To determine if TAG72-BBζ CAR T cells demonstrate selective activity against TAG72-positive cancer cells, the TAG72-BBζ CAR T cells were grown in presence of either TAG72-positive or TAG72-negative ovarian cancer cells and the percentage of ovarian cancer cells killed was quantified.

### Results

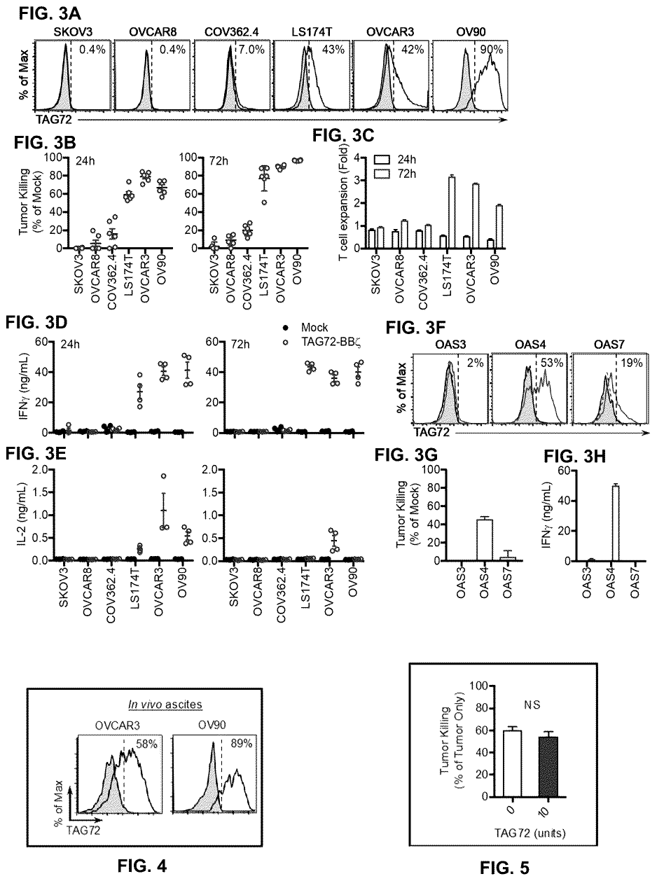

As a first step toward evaluating TAG72-BBζ CAR T cells selective activity—including targeting and conferring cell death of target cells—against TAG72-positive cancer cells, TAG72 expression on human ovarian cancer cell lines, including SKOV3, OVCAR8, COV362.4, OVCAR3, OV90, as well as the TAG72+ colon cancer line, LS174T, was evaluated to identify a TAG72-positive cancer cell line. Prior studies have demonstrated expression of TAG72 by immunohistochemistry of ovarian tumor patient samples and by western blotting of human ovarian cancer cell lines (Chauhan S C, Vinayek N, Maher D M, Bell M C, Dunham K A, Koch M D, Lio Y, Jaggi M. Combined staining of TAG-72, MUC1, and CA125 improves labeling sensitivity in ovarian cancer: antigens for multi-targeted antibody-guided therapy. The journal of histochemistry and cytochemistry: official journal of the Histochemistry Society. 2007; 55(8):867-75; Ponnusamy M P, Venkatraman G, Singh A P, Chauhan S C, Johansson S L, Jain M, Smith L, Davis J S, Remmenga S W, Batra S K. Expression of TAG-72 in ovarian cancer and its correlation with tumor stage and patient prognosis. Cancer letters. 2007; 251(2):247-57). By flow cytometry, TAG72 was expressed on OVCAR3 cells (approximately 42%) and to a greater extent on OV90 cells (approximately 90%), with very low levels detected on COV362.4 cells (FIG. 3A). TAG72 was absent on SKOV3 and OVCAR8 cells. Immunofluorescence staining of tumor cells confirmed TAG72 expression and cellular localization on the cell surface as well as intracellularly. Notably, higher expression of TAG72 on OVCAR3 and OV90 cells harvested from the ascites of tumor-bearing animals was observed as compared to in vitro cultured cells (FIG. 4).

To assess antigen-dependent activity of our TAG72-BBζ CAR T cells, co-cultured assays with TAG72-positive and -negative ovarian tumor targets were conducted at an E:T ratio between 1:1 and 1:2 to determine their killing potential. After 24 hours, antigen-specific T cell-mediated killing activity was evident with TAG72-BBζ CAR T cells relative to Mock T cells (FIG. 3B). Amongst TAG72− expressing targets, an average of 59% LS174T, 79% OVCAR3, and 67% OV90 cells were killed. After 72 hours, killing of the same tumor lines increased to 77%, 90%, and 97%, respectively. TAG72− BBζ CAR T cells showed minimal killing of TAG72-negative or low expressing SKOV3, OVCAR8, and COV362.4 cells. At 72 hours, TAG72-BBζ CAR T cell expansion against TAG72-positive tumor cells was 2- to 3-fold (FIG. 3C). Similar tumor killing was observed at lower E:T ratios of 1:10, demonstrating the potent killing ability of TAG72-BBζ CAR T cells. The tumor killing ability of TAG72-BBζ CAR T cells was minimally impacted by soluble TAG72 shed from tumor cells (FIG. 5). Cytokine production from CAR T cells was measured as an additional measure of T cell activity. IFNγ and IL-2 cytokine production was observed only when TAG72-BBζ CAR T cells were co-cultured with antigen-positive tumor targets, OVCAR3, LS174T, and OV90 (FIGS. 3D and 3E). While IL-2 production peaked at early time points (T=24 hour) and was detectable only against OVCAR3 at later time points (T=72 hours), in contrast IFNγ levels exhibited elevated levels over the full 72 hours.

### Example 3: Validation that TAG72-BBζ CAR T Cells Selectively Target TAG72-Positive Cells from Ovarian Cancer Ascites In Vitro

To further confirm TAG72 as an ovarian cancer CAR target and the anti-tumor activity of our TAG72-BBζ CAR T cells, TAG72-BBζ CAR T cells were grown in presence of human ovarian cancer ascites from three patients (OAS3, OAS4, OAS7).

### Results

Freshly thawed ascites from OAS3, OAS4, and OAS7 expressed 62%, 80%, and 67% TAG72, respectively, by flow cytometry, but after 72 hours in culture, was reduced to 2%, 53%, and 19%, respectively (FIG. 3F). Without wishing to be bound to a particular theory, the reduction in TAG72 expression may reflect an influence of ex vivo culturing conditions on maintenance of TAG72 expression (Horan Hand P, Colcher D, Salomon D, Ridge J, Noguchi P, Schlom J. Influence of spatial configuration of carcinoma cell populations on the expression of a tumor-associated glycoprotein. Cancer research. 1985; 45(2):833-40). TAG72-BBζ CAR T cells exhibited cytolytic activity after 72 hours of co-culture with ascites, and showed potent and selective CAR-mediated killing of the TAG72-positive OAS4 and OAS7 cells, with no detectable anti-tumor activity against the TAG72-negative OAS3 cells (FIG. 3G). TAG72-BBζ CAR T cells produced IFNγ and IL-2 against OAS4, but not OAS3 and OAS7 cells (FIG. 3H). These data suggest that TAG72-CAR T cells selectively target TAG72-positive cells from ovarian cancer ascites in vitro.

### Example 4: Validation that TAG72-BBζ CAR T Cells Delivered Locally to Ovarian Ascites In Vivo in a Mouse Model Exhibit Potent Anti-Tumor Activity and Confer Extended Lifespan to the Mice

To evaluate the therapeutic potential of the TAG72-BBζ CAR T cells in vivo, the ability of TAG72-BBζ CAR T cells to selectively target TAG72-positive OVCAR3 tumors in immune compromised NSG mice was tested; this mouse model mimics mimic peritoneal ovarian tumors observed in late-stage human disease. The TAG72-BBζ CAR T cells were delivered by intraperitoneal (i.p.) injection.

### Results

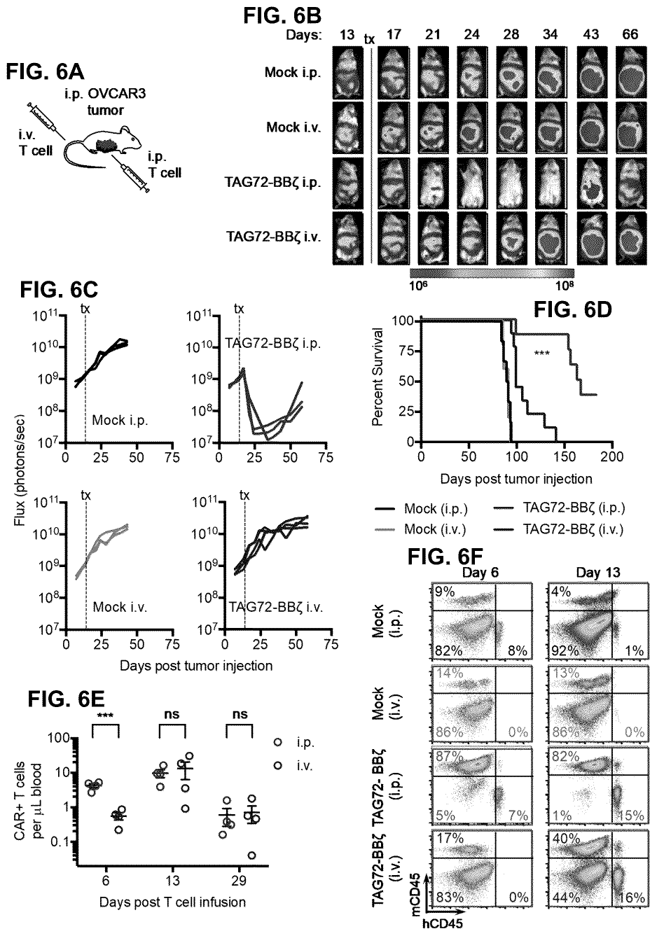

OVCAR3 cells were lentivirally transduced to express eGFP/ffluc to allow for tracking of tumor growth via non-invasive optical imaging. At 14 days post tumor i.p. injection, mice were treated with Mock or TAG72-BBζ CAR T cells (5.0×10⁶) by systemic intravenous (i.v.) or regional i.p. delivery (FIG. 6A). Rapid anti-tumor effects were observed in mice treated with TAG72-BBζ CAR T cells via regional i.p. delivery, reaching a maximal anti-tumor response 1-2 weeks following treatment (FIGS. 6B and 6C). In comparison to regional delivery, i.v. delivery of TAG72-BBζ CAR T cells showed limited anti-tumor responses. Anti-tumor responses in mice were durable for 3-4 weeks, but ultimately tumor recurrences were observed in mice. Regional i.p. delivery of TAG72-BBζ CAR T cells significantly extended survival of mice, with limited benefits observed by i.v. delivery (FIG. 6D).

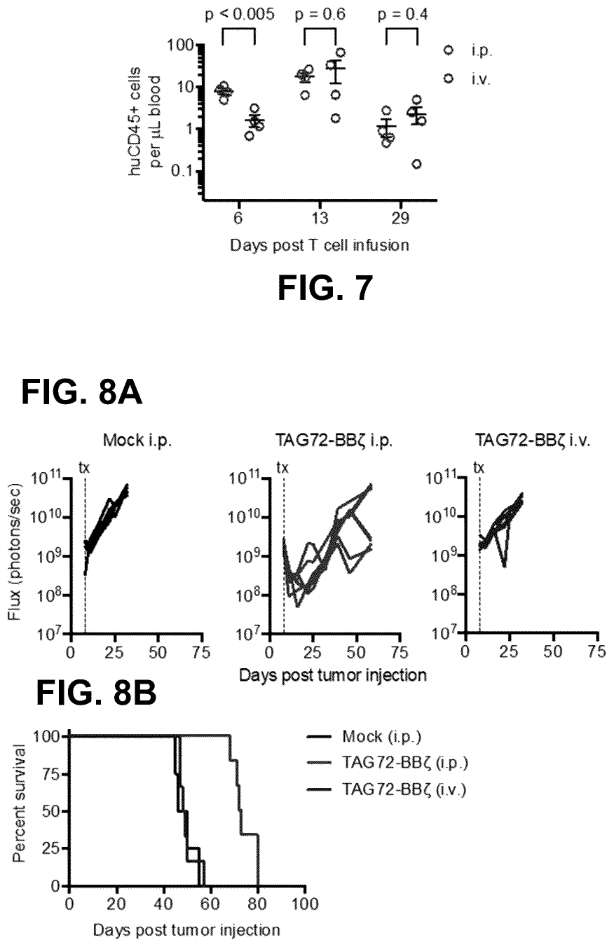

To address potential differences observed between i.p. and i.v. therapy, CAR T cells in the blood and ascites of mice were quantified. Strikingly, appreciable numbers of CAR T cells (huCD45+CD19t+) were found in the blood of mice 6 days post i.p. treatment, with more than 5-fold fewer CAR T cells in the blood of i.v. treated mice at the same time point (FIG. 6E and FIG. 7). However, equivalent numbers of CAR T cells were observed in the blood of i.p. and i.v. treated mice at later time points, expanding from 1-2 weeks, with significant reductions at 4 weeks post treatment. CAR T cells in the ascites of treated mice continued to be present at the site of tumors at day 6 post i.p. treatment, with no detectable CAR T cells in i.v. treated mice at the same time point. However, at day 13 post treatment, similar levels of CAR T cells were observed in mice treated i.v. and i.p. (FIG. 6F). Without wishing to be bound to a particular theory, these data collectively suggest that CAR T cells eventually reached the tumor following i.v. delivery but with delayed kinetics compared with i.p. delivery, which was likely in part responsible for the lack of observed therapy by this route of administration. CD45-negative cells, likely majority being OVCAR3 tumor cells, were significantly depleted in i.p. TAG72-BBζ CAR T cell treated mice, but not i.p. or i.v. Mock T cell or i.v. TAG72-BBζ CAR T cell treated mice. These data support regional intraperitoneal delivery of TAG72-CAR T cells as an effective method of targeting peritoneal ovarian tumors in mice.

### Example 5: Validation that TAG72-BBζ CAR T Cells Selectively Target TAG72-Positive Cells in OV90 i.p. Model and Comparison of Efficacy for TAG72-BBζ CAR T Cells Administered as Single Versus Multiple Dosing Regimen

To evaluate efficacy of TAG72-BBζ CAR T cells to selective target TAG72-positive cells in the OV90 i.p. model, TAG72-BBζ CAR T cells were delivered either as single or multiple repeat doses and tumor size was evaluated over time.

### Results

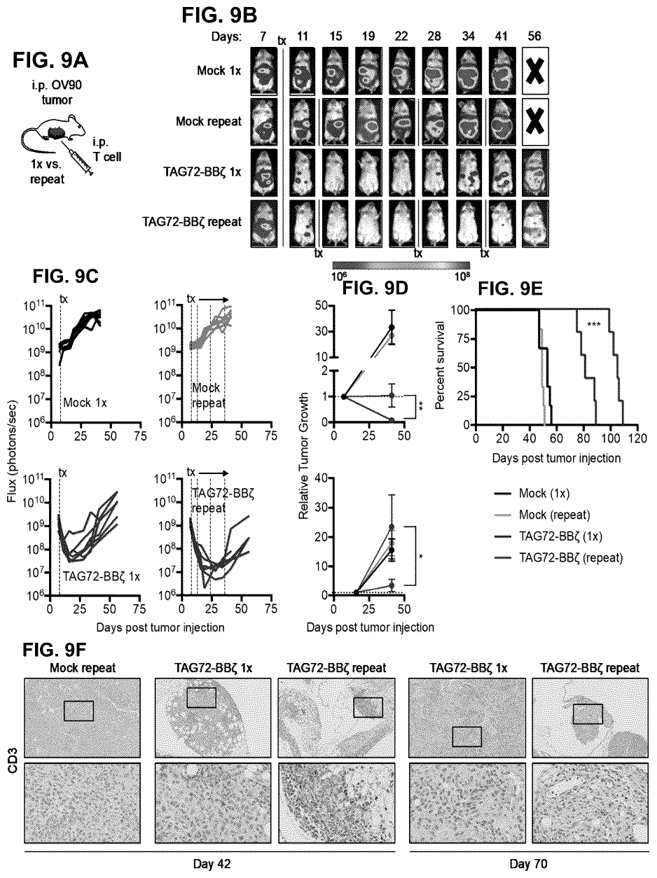

Notably, the OV90 i.p. model exhibits more uniform TAG72 expression in vitro compared with OVCAR3 (FIG. 3A). Regional CAR T cell delivery in the OV90 i.p. model showed selective targeting of TAG72 cells compared to the OVCAR3 model, i.p. In contrast, i.v. TAG72-BBζ CAR T cell treatment failed to show anti-tumor efficacy in the OV90 model (FIG. 8). Overall survival was only delayed by approximately 25 days in this model with i.p. delivery of TAG72-BBζ CAR T cells (FIG. 8), likely owing to the aggressive nature of this model. Given this observation, the efficacy of repeat TAG72-BBζ CAR T cell dosing compared with a single dose was evaluated and found to improve therapeutics responses (FIG. 9A). Compared with a single dose of TAG72-BBζ CAR T cells, repeat dosing over the course of one month demonstrated more durable anti-tumor responses in the OV90 model (FIGS. 9B and 9C). When plotted as relative tumor growth kinetics, repeat dosing promoted more extensive tumor regression as well as more durable control of tumors compared with single dosing (FIG. 9D).

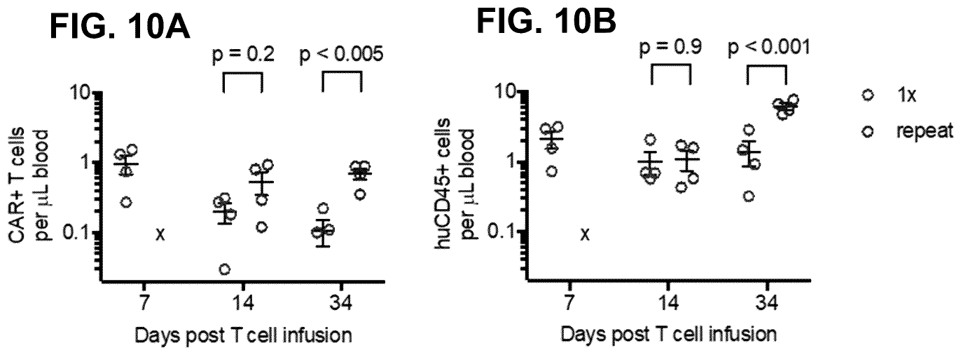

In this study, the overall survival was extended significantly in mice that received repeat doses of TAG72-BBζ CAR T cells (55 day benefit) compared with a single dose (30 day benefit) (FIG. 9E). Greater T cell numbers were observed in peritoneal tumors of mice with repeat treatment (FIG. 9F). Importantly, however, reduced numbers, expansion and persistence of CAR T cells in the blood of OV90-bearing mice was observed compared with the OVCAR3 model (FIG. 10). Without wishing to be bound to a particular theory, these results suggest that this more aggressive tumor model may also harbor suppressive mechanisms that hamper T cell function and overall CAR T cell efficacy. Collectively, these data demonstrate potent anti-tumor activity of TAG72-BBζ CAR T cells in ovarian cancer xenograft models, and also suggest that repeat dosing of regionally delivered CAR T cells may provide greater control of tumors compared with a single dose.

### Example 6: Determination that Tumor Recurrences Following TAG72-CAR T Cell Therapy Exhibit Antigen Escape

Given that TAG72-BBζ CAR T cells in previous Examples were observed to reduce prior to tumor recurrences, the expression of TAG72 in tumors was quantified over time to determine if loss of TAG72 expression correlates with reduced TAG72-BBζ CAR T cell numbers.

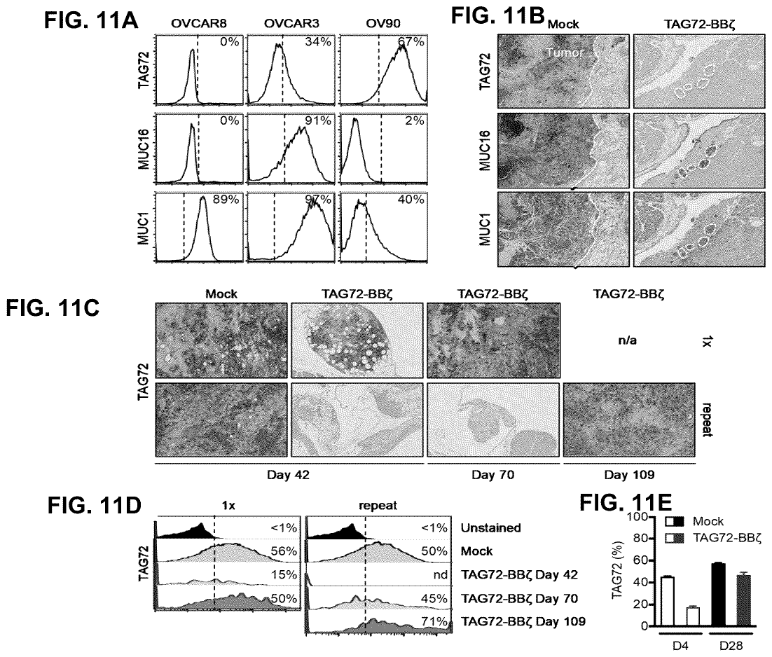

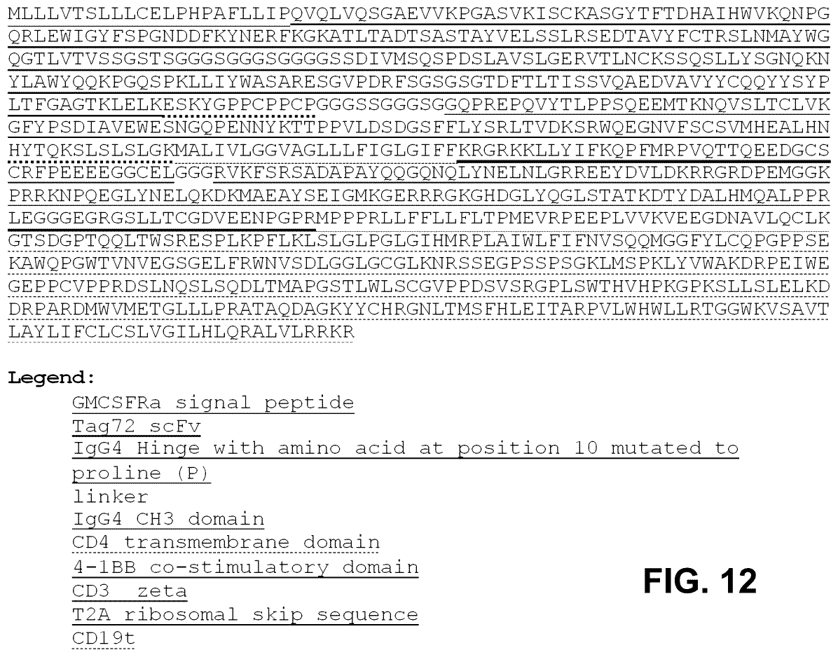

Results One of the major resistance mechanisms to CAR T cell therapy is the tumor antigen heterogeneity that exists in solid tumors that promotes eventual antigen loss or escape (Chen N, Li X, Chintala N K, Tano Z E, Adusumilli P S. Driving CARs on the uneven road of antigen heterogeneity in solid tumors. Current opinion in immunology. 2018; 5 1:103-10). Given that the loss of CAR T cells in the two in vivo models (of previous Examples) preceded tumor recurrences, potentially loss of TAG72 expression in tumors occurs correlates with loss of CAR T cells. To evaluate the former, the expression of TAG72 in tumors from Mock and TAG72-BBζ CAR T cell treated mice was measured over time pre- and post-therapy. Since TAG72, MUC1, and MUC16 have all been identified as potential targets in ovarian cancer, the expression of these cell surface antigens on TAG72-negative OVCAR8, and TAG72-positive OVCAR3 and OV90 cells was quantified. OVCAR8 appeared to only express low levels of MUC1, and was absent for TAG72 and MUC16, while OVCAR3 expressed all three antigens at varying levels, and OV90 showed low expression of MUC1 and was absent for MUC16 (FIG. 11A). Therefore, the expression of these antigens in OVCAR3 tumors from mice treated with Mock or TAG72-BBζ CAR T cells was quantified. At twelve weeks post T cell infusion, tumors from Mock-treated mice showed heterogeneous expression of TAG72 (similar to flow cytometry analysis of the cell line), MUC16, and MUC1 (FIG. 11B). However, tumor recurrences at early time points from mice treated with TAG72-BBζ CAR T cells showed a dramatic reduction in TAG72 expression, while maintaining expression of MUC16 and MUC1. Similarly, repeat treatment of TAG72-BBζ CAR T cells in the OV90 tumor model also showed a reduction in TAG72 expression in early recurrent tumors following treatment (FIG. 11E). Notably, the expression of TAG72 was detected at high levels in tumor recurrences at later time points, in solid tumors as well as in ascites (FIGS. 11C and 11D). To further confirm this finding in vitro, the expression of TAG72 in tumor cells following CAR T cell co-culture was quantified and found to be reduced compared to tumor cells that grew out in the absence of co-culture with CAR T cells (FIG. 11E). In total, these data suggest that antigen escape likely plays a key role in tumor recurrences following TAG72-BBζ CAR T cell therapy.

### Example 7: Validation that TAG72-BBζ CAR T Cells Selectively Target and Exhibit Activation Against TAG72-Positive Ovarian Cancer Cells In Vitro

To determine if humanized TAG72-BBζ CAR T cells also effectively kill TAG72-positive cancer cells, humanized TAG72-BBζ CAR T cells were grown in presence of TAG72-positive ovarian cancer cells and the percentage of ovarian cancer cells killed was quantified.

### Results

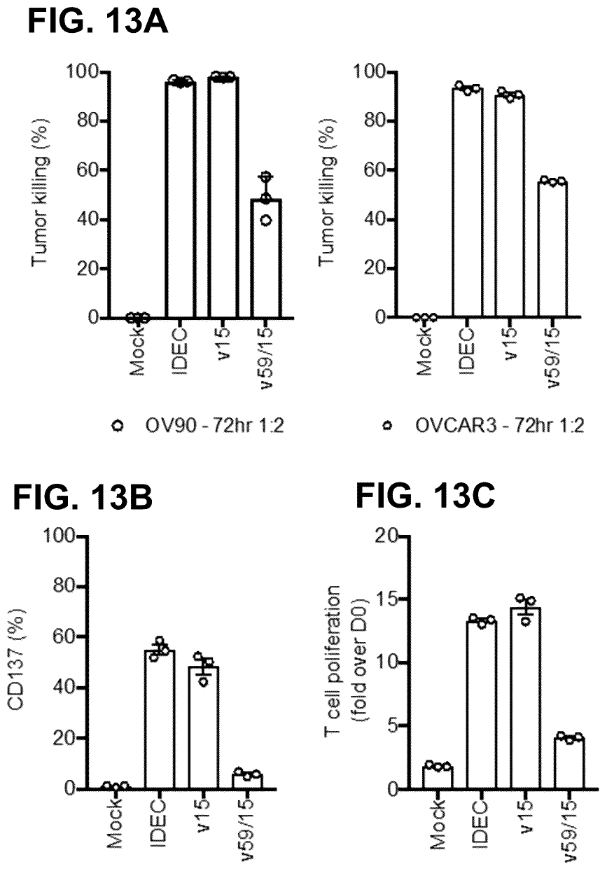

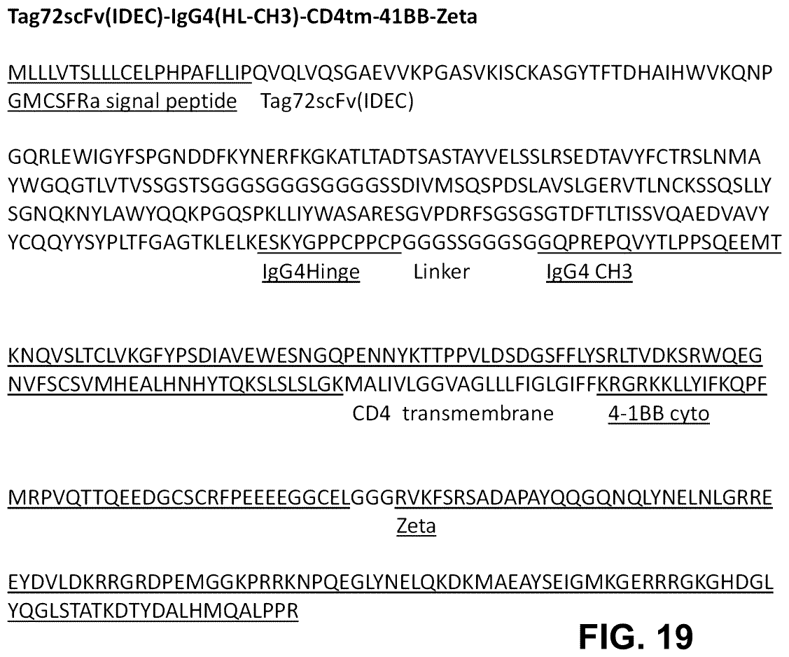

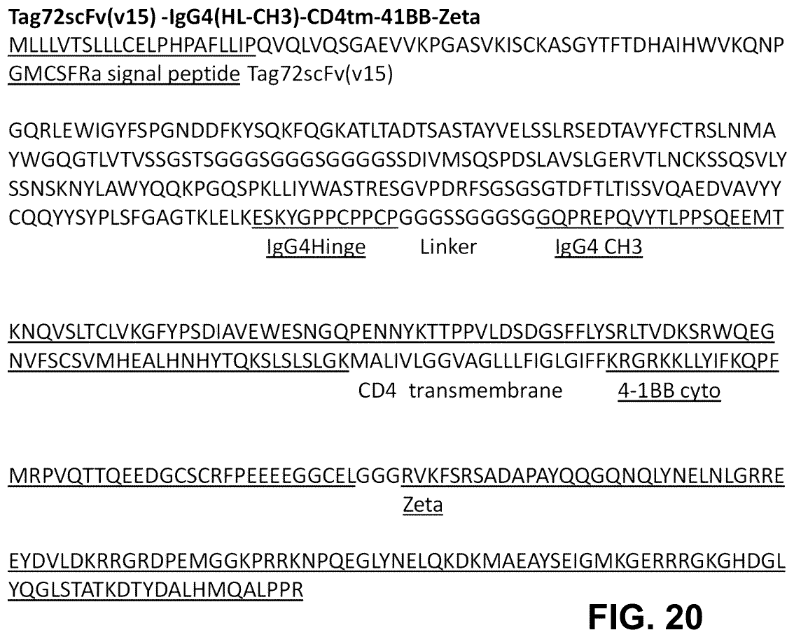

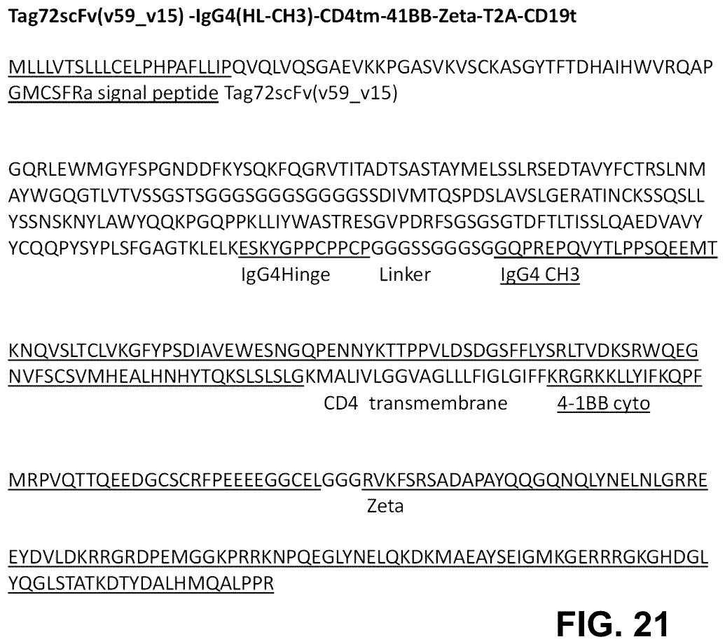

A series of representative 4-1BB co-stimulated CAR T cells that expresses either the IDEC, V15, or a combined V59/V15 antigen-binding domain (scFv) from humanized variants of anti-TAG-72 antibody clone CC49 (IDEC-TAG72-BBz, V15-TAG72-BBz, or V59/15-TAG72-BBz; FIGS. 19-21 (shown without the T2A and CD1t sequences present at the carboxy terminus) were created. These CARs all utilize the same extracellular domain (composed of IgG4 hinge with a mutation to P at amino acid 10 of the hinge; a linker having the sequence GGGSSGGGSG and the human IgG CH3 domain), a CD4 transmembrane domain, and a 4-1BB intracellular co-stimulatory signaling domain. These humanized TAG72-BBζ CAR T cells were grown in presence of either OV90 or OVCAR3 ovarian cancer cells and the percentage of ovarian cancer cells killed was quantified. In vitro, both the IDEC and V15 TAG72-BBz CAR T cells show equivalent potent T cell-mediated antigen-dependent cytotoxicity, activation, and T cell proliferation following exposure to TAG72-expressing ovarian cancer cell lines (FIG. 13A-13C). The V59/15 TAG72-BBz CARs showed little activity in this assay and was removed from further experiments.

### Example 8: Validation that Humanized TAG72-BBζ CAR T Cells Selectively Target TAG72-Positive Cells in OV90 i.p. Model and Comparison of Efficacy for TAG72-BBζ CAR T Cells Administered as Single Versus Multiple Dosing Regimen

To evaluate efficacy of humanized TAG72-BBζ CAR T cells to selective target TAG72-positive cells in the OV90 i.p. model, humanized TAG72-BBζ CAR T cells were delivered either as single or multiple repeat doses and tumor size was evaluated over time.

### Results

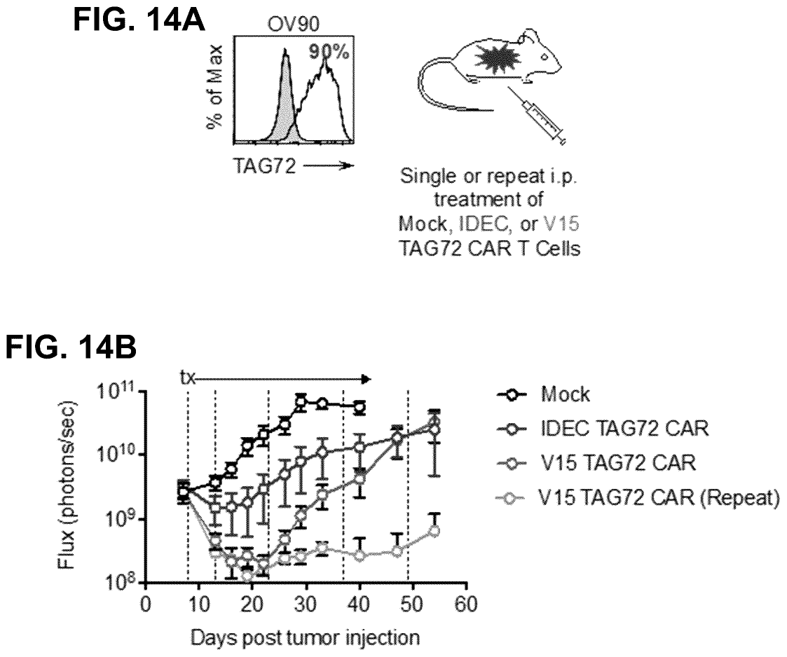

Endogenous expression of TAG72 antigen on OV90 tumor cell line was determined by flow cytometry. OV90-ffluc cells were injected into the intraperitoneal (i.p.) cavity of NSG mice and tracked by bioluminescent imaging and reported as flux (photos/sec). At 8 days post tumor injection, either a single or repeat dose of 5.0×10⁶ Mock, IDEC, or V15 variants of TAG72 CAR T cells administered regionally into the i.p. cavity of tumor-bearing mice (FIG. 14A). Tumor burden of single or repeat T cell-treated mice was quantified by bioluminescent imaging. Dashed vertical lines indicate time points of initial and repeated treatment with T cells. Interestingly, we show using in vivo ovarian tumor models that regional intraperitoneal treatment with V15-TAG72-BBz reduces tumor burden of antigen-positive targets (OV90 engrafted tumors) to a greater extent than IDEC-TAG72-BBz CARs (FIGS. 14A-14B). Given this observation, the efficacy of repeat TAG72-BBζ CAR T cell dosing compared with a single dose was evaluated and found to improve therapeutics responses (FIG. 14B). Compared with a single dose of TAG72-BBζ CAR T cells, repeat dosing over the course of over 50 days demonstrated more durable anti-tumor responses in the OV90 model (FIG. 14B).

### Example 9: Validation that Humanized TAG72-BBζ CAR T Cells Selectively Target TAG72-Positive Cells in OVCAR3 i.p. Model and Comparison of Persistence for TAG72-BBζ CAR T Cells Administered as a Single Dosing Regimen

To evaluate efficacy of humanized TAG72-BBζ CAR T cells to selective target TAG72-positive cells in the OVCAR3 i.p. model, humanized TAG72-BBζ CAR T cells were delivered as a single dose and tumor size was evaluated over time.

### Results

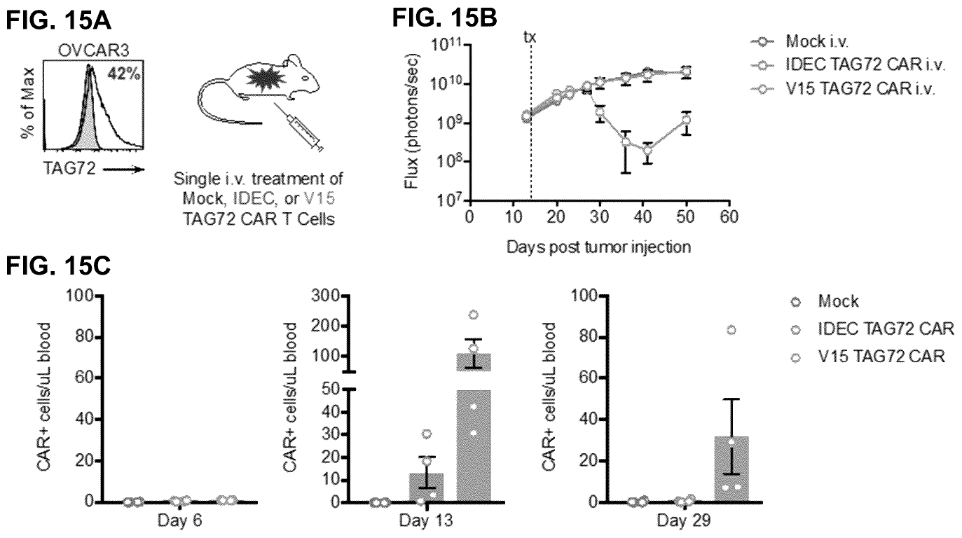

Endogenous surface TAG72 expression was analyzed by flow cytometry on OVCAR3 tumor cells. OVCAR3-ffluc tumors were then injected into the i.p. cavity of NSG mice, that were treated i.v. with a single dose of 5.0×10⁶ Mock, IDEC, or V15 variant TAG72 CAR T cells (FIG. 15A). Tumor burden of single dose treated mice was quantified by bioluminescent imaging and reported as flux (photos/sec). Dashed vertical lines indicate time point of treatment with T cells. Interestingly, intravenously (i.v.) administered V15-TAG72-BBz CAR T cells, but not IDEC-TAG72-BBz CAR T cells, are able to mediate a robust anti-tumor response against OVCAR3 tumor bearing mice (FIG. 15B). This anti-tumor response in vivo of the V15-TAG72-BBz CARs was in part mediated by an increased proliferation compared to IDEC-TAG72-BBz, thus increasing the longevity of its response (FIG. 15C).

### Example 10: Determination that Humanized TAG72-BBζ CAR T Cells Design Affects Tumor Killing, T Cell Proliferation, Activation, Exhaustion, and Cytokine Production

To evaluate the design of humanized TAG72 CAR T cells, a series of representative TAG72 CAR T cells was created featuring the V15 scFv and varying the linker, transmembrane, and costimulatory domains.

### Results

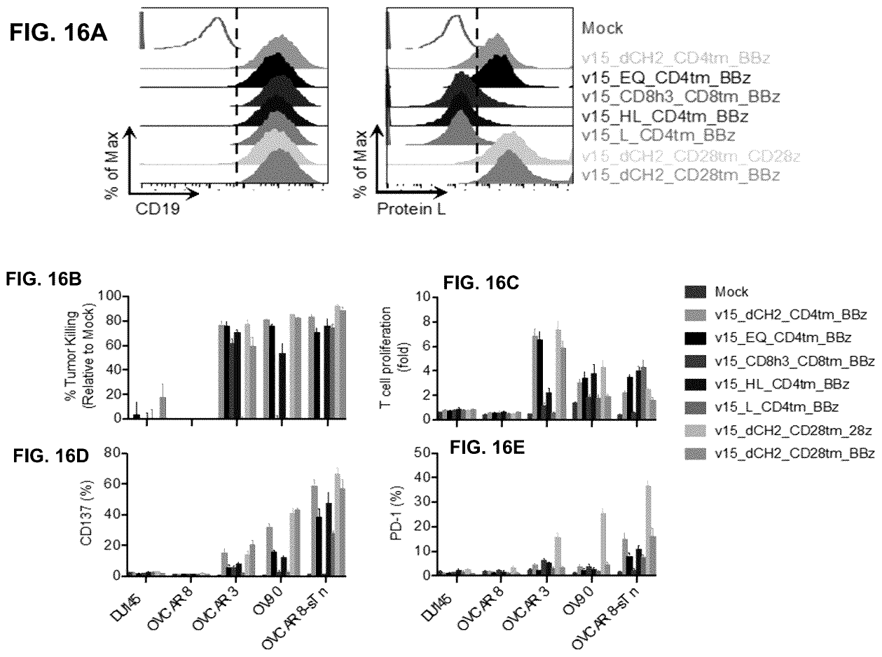

All seven representative humanized TAG72-CAR T cells variants with the V15 scFv clone exhibited CAR expression stability (FIG. 16A). In an in vitro tumor killing activity, humanized TAG72− CAR T cells were grown in presence of either TAG72-positive (OVCAR3, OV90, and OVCAR8-sTn) or TAG72-negative (DU145, OVCAR8) ovarian cancer cells and the percentage of ovarian cancer cells killed was quantified. All seven representative humanized TAG72-CAR T cells variants showed potent and selective CAR-mediated killing of the TAG72− positive OVCAR3, OV90, and OVCAR8-sTn cells, with no detectable anti-tumor activity against the TAG72-negative DU145 and OVCAR8 cells (FIG. 16B). T cell proliferation varied and was higher in the TAG72-positive OVCAR3, OV90, and OVCAR8-sTn cells than in the TAG72-negative DU145 and OVCAR8 cells (FIG. 16C). CD137+ activation indicator showed that the representative humanized TAG72-CAR T cells variants varied and was higher in the TAG72-positive OVCAR3, OV90, and OVCAR8-sTn cells than in the TAG72-negative DU145 and OVCAR8 cells (FIG. 16D). PD-1+ exhaustion indicator (72 hours) of CAR T cells against TAG72-negative (DU145, OVCAR8), and TAG72-positive (OVCAR3, OV90, and OVCAR8-sTn) expressing tumor cells (FIG. 16E).

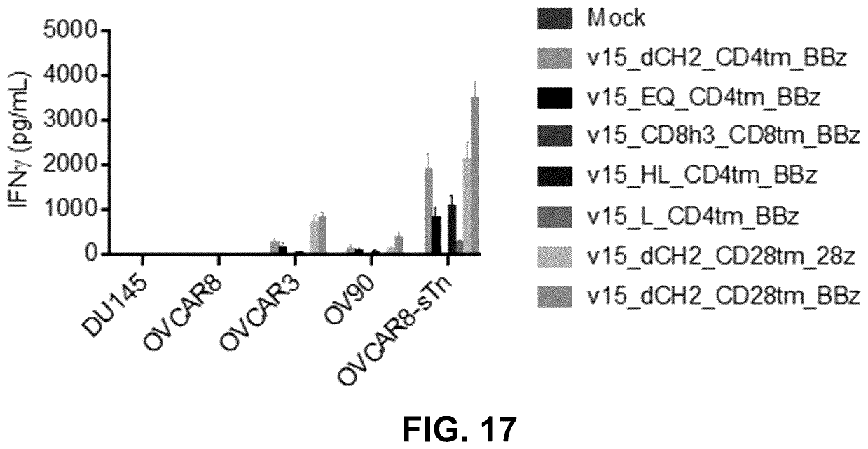

Varying V15-CAR design also impacts in vitro cytokine production of TAG72-CAR T cells. In vitro IFNγ production (24 hours), of CAR T cells against TAG72-negative (DU145, OVCAR8), and TAG72-positive (OVCAR3, OV90, OVCAR8-sTn) expressing tumor cells. While CARs with a CD28tm-BBz construct shows similar anti-tumor activity compared with the CD4tm-BBz construct, the CD28tm confers greater cytokine production in some TAG72-positive tumor cells (FIG. 17).

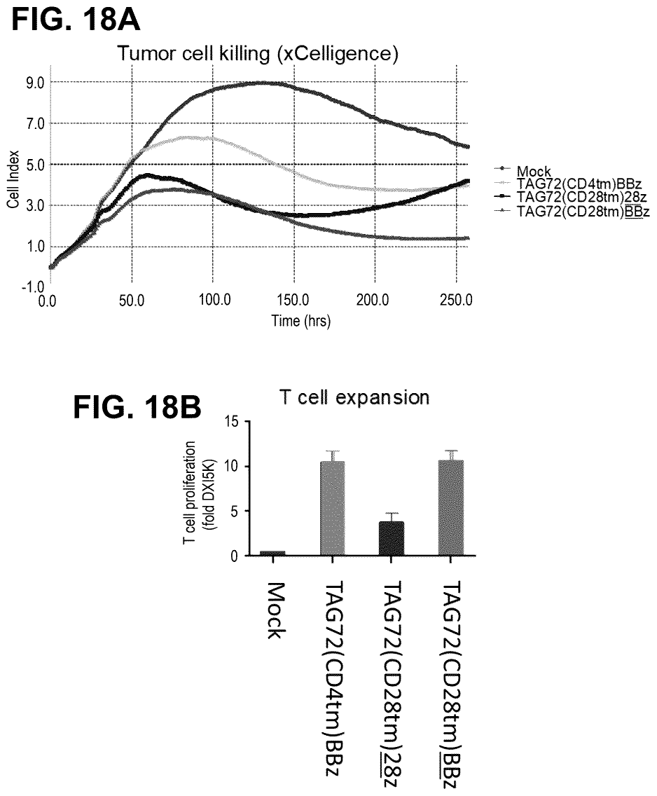

A real-time cytotoxicity assay was performed using xCelligence technology with OV90 cells and a few representative humanized TAG72-CAR T cells variants. The four T cells populations were plated at an effector to target ratio of 1-to-20 and observed for 10 days. Cell Index is indicative of live tumor count. All three representative humanized TAG72-CAR T cells variants demonstrated potent anti-tumor activity in this long-term killing assay (FIG. 18A). At the long-term killing assay endpoint, remaining cells were collected and analyzed by flow cytometry. T cell expansion was demonstrated for all three representative humanized TAG72-CAR T cells variants (FIG. 18B).

What is claimed is:

1. A nucleic acid molecule comprising a nucleotide sequence encoding a chimeric antigen receptor (CAR), wherein the chimeric antigen receptor comprises: an scFv targeting Tag-72, a spacer, a transmembrane domain, a CD28 or 41-BB co-stimulatory domain, and a CD3 ζ signaling domain.

2. The nucleic acid molecule of claim 1, wherein the transmembrane domain is selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications.

3. The nucleic acid molecule of claim 1, wherein the TAG72 scFV is selected from IDEC, V15 and V59_V15.

4. The nucleic acid molecule of claim 1, wherein the transmembrane domain is a CD4 transmembrane domain or variant thereof having 1-5 amino acid modifications.

5. The nucleic acid molecule of claim 1, wherein the transmembrane domain is a CD4 transmembrane domain.

6. The nucleic acid molecule of claim 1, wherein the chimeric antigen receptor comprises a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications,

7. The nucleic acid molecule of claim 1, wherein the spacer region comprises an amino acid sequence selected from the group consisting of SEQ ID NOs: 2-12 or a variant thereof having 1-5 amino acid modifications.

8. The nucleic acid molecule of claim 1, wherein the spacer comprises an IgG hinge region.

9. The nucleic acid molecule of claim 1, wherein the spacer comprises 10-50 amino acids.

10. The nucleic acid molecule of claim 1, wherein the 4-1BB costimulatory domain comprises the amino acid sequence of SEQ ID NO: 24 or a variant thereof having 1-5 amino acid modifications.

11. The nucleic acid molecule of claim 1, wherein the CD3ζ signaling domain comprises the amino acid sequence of SEQ ID NO:21.

12. The nucleic acid molecule of claim 1, wherein a linker of 3 to 15 amino acids is located between the 4-1BB costimulatory domain and the CD3 ζ signaling domain or variant thereof.

13. The nucleic acid molecule of claim 1, wherein the CAR comprises the amino acid sequence of SEQ ID NO: 29 or a variant thereof having 1-5 amino acid modifications.

14. The nucleic acid molecule of claim 7, wherein the scFv comprises the amino acid sequence of SEQ ID NO:1, 31 or 32.

15. An expression vector comprising the nucleic acid molecule of claim 1.

16. A viral vector comprising the nucleic acid molecule of claim 1,

17. A population of human T cells transduced by a vector comprising the nucleic acid molecule of claim 1.

18. The population of human T cells of claim 17, wherein the population of human T cells comprise central memory T cells.

19. A method of treating solid tumor in a patient comprising administering a population of autologous or allogeneic human T cells transduced by a vector comprising the nucleic acid molecule of claim 1, wherein the solid tumor comprises cells expressing Tag-72.

20. The method of claim 19, wherein the chimeric antigen receptor is administered locally or systemically.

21. The method of claim 1, wherein the TAG72-expressing cells are ovarian cancer cells.

22. The method of claim 19, wherein the chimeric antigen receptor is administered by single or repeat dosing.


SEQUENCE LISTING (41 sequences)

SEQ ID NO: 1 - protein, 246 aa, Homo sapiens
QVQLVQSGAEVVKPGASVKISCKASGYTFTDHAIHWVKQNPGQRLEWIGYFSPGNDDFKYNERFKGKATLTADTSASTAYVELSSLRSEDTAVYFCTRSLNMAYWGQGTLVTVSSGSTSGGGSGGGSGGGGSSDIVMSQSPDSLAVSLGERVTLNCKSSQSLLYSGNQKNYLAWYQQKPGQSPKLLIYWASARESGVPDRFSGSGSGTDFTLTISSVQAEDVAVYYCQQYYSYPLTFGAGTKLELK

SEQ ID NO: 2 - protein, 10 aa, Homo sapiens
GGGSSGGGSG

SEQ ID NO: 3 - protein, 12 aa, Homo sapiens
ESKYGPPCPPCP

SEQ ID NO: 4 - protein, 12 aa, Homo sapiens
ESKYGPPCPSCP

SEQ ID NO: 5 - protein, 22 aa, Homo sapiens
ESKYGPPCPPCPGGGSSGGGSG

SEQ ID NO: 6 - protein, 39 aa, Homo sapiens
IEVMYPPPYLDNEKSNGTIIHVKGKHLCPSPLFPGPSKP

SEQ ID NO: 7 - protein, 48 aa, Homo sapiens
AKPTTTPAPRPPTPAPTIASQPLSLRPEACRPAAGGAVHTRGLDFACD

SEQ ID NO: 8 - protein, 45 aa, Homo sapiens
TTTPAPRPPTPAPTIASQPLSLRPEACRPAAGGAVHTRGLDFACD

SEQ ID NO: 9 - protein, 129 aa, Homo sapiens
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, Homo sapiens
ESKYGPPCPSCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, Homo sapiens
ESKYGPPCPPCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Homo sapiens
LCYLLDGILFIYGVILTALFL

SEQ ID NO: 14 - protein, 27 aa, Homo sapiens
FWVLVVVGGVLACYSLLVTVAFIIFWV

SEQ ID NO: 15 - protein, 28 aa, Homo sapiens
MFWVLVVVGGVLACYSLLVTVAFIIFWV

SEQ ID NO: 16 - protein, 22 aa, Homo sapiens
MALIVLGGVAGLLLFIGLGIFF

SEQ ID NO: 17 - protein, 21 aa, Homo sapiens
IYIWAPLAGTCGVLLLSLVIT

SEQ ID NO: 18 - protein, 23 aa, Homo sapiens
IYIWAPLAGTCGVLLLSLVITLY

SEQ ID NO: 19 - protein, 24 aa, Homo sapiens
IYIWAPLAGTCGVLLLSLVITLYC

SEQ ID NO: 20 - protein, 27 aa, Homo sapiens
IISFFLALTSTALLFLLFFLTLRFSVV

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Homo sapiens
RSKRSRLLHSDYMNMTPRRPGPTRKHYQPYAPPRDFAAYRS

SEQ ID NO: 23 - protein, 41 aa, Homo sapiens
RSKRSRGGHSDYMNMTPRRPGPTRKHYQPYAPPRDFAAYRS

SEQ ID NO: 24 - protein, 42 aa, Homo sapiens
KRGRKKLLYIFKQPFMRPVQTTQEEDGCSCRFPEEEEGGCEL

SEQ ID NO: 25 - protein, 42 aa, Homo sapiens
ALYLLRRDQRLPPDAHKPPGGGSFRTPIQEEQADAHSTLAKI

SEQ ID NO: 26 - protein, 923 aa, Homo sapiens
MLLLVTSLLLCELPHPAFLLIPQVQLVQSGAEVVKPGASVKISCKASGYTFTDHAIHWVKQNPGQRLEWIGYFSPGNDDFKYNERFKGKATLTADTSASTAYVELSSLRSEDTAVYFCTRSLNMAYWGQGTLVTVSSGSTSGGGSGGGSGGGGSSDIVMSQSPDSLAVSLGERVTLNCKSSQSLLYSGNQKNYLAWYQQKPGQSPKLLIYWASARESGVPDRFSGSGSGTDFTLTISSVQAEDVAVYYCQQYYSYPLTFGAGTKLELKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 27 - protein, 24 aa, Homo sapiens
LEGGGEGRGSLLTCGDVEENPGPR

SEQ ID NO: 28 - protein, 354 aa, Homo sapiens
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 29 - protein, 576 aa, Homo sapiens
MLLLVTSLLLCELPHPAFLLIPQVQLVQSGAEVVKPGASVKISCKASGYTFTDHAIHWVKQNPGQRLEWIGYFSPGNDDFKYNERFKGKATLTADTSASTAYVELSSLRSEDTAVYFCTRSLNMAYWGQGTLVTVSSGSTSGGGSGGGSGGGGSSDIVMSQSPDSLAVSLGERVTLNCKSSQSLLYSGNQKNYLAWYQQKPGQSPKLLIYWASARESGVPDRFSGSGSGTDFTLTISSVQAEDVAVYYCQQYYSYPLTFGAGTKLELKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 576 aa, Homo sapiens; identical to SEQ ID NO: 29

SEQ ID NO: 31 - protein, 576 aa, Homo sapiens
MLLLVTSLLLCELPHPAFLLIPQVQLVQSGAEVVKPGASVKISCKASGYTFTDHAIHWVKQNPGQRLEWIGYFSPGNDDFKYSQKFQGKATLTADTSASTAYVELSSLRSEDTAVYFCTRSLNMAYWGQGTLVTVSSGSTSGGGSGGGSGGGGSSDIVMSQSPDSLAVSLGERVTLNCKSSQSVLYSSNSKNYLAWYQQKPGQSPKLLIYWASTRESGVPDRFSGSGSGTDFTLTISSVQAEDVAVYYCQQYYSYPLSFGAGTKLELKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 576 aa, Homo sapiens
MLLLVTSLLLCELPHPAFLLIPQVQLVQSGAEVKKPGASVKVSCKASGYTFTDHAIHWVRQAPGQRLEWMGYFSPGNDDFKYSQKFQGRVTITADTSASTAYMELSSLRSEDTAVYFCTRSLNMAYWGQGTLVTVSSGSTSGGGSGGGSGGGGSSDIVMTQSPDSLAVSLGERATINCKSSQSLLYSSNSKNYLAWYQQKPGQPPKLLIYWASTRESGVPDRFSGSGSGTDFTLTISSLQAEDVAVYYCQQPYSYPLSFGAGTKLELKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 246 aa, Homo sapiens
QVQLVQSGAEVVKPGASVKISCKASGYTFTDHAIHWVKQNPGQRLEWIGYFSPGNDDFKYSQKFQGKATLTADTSASTAYVELSSLRSEDTAVYFCTRSLNMAYWGQGTLVTVSSGSTSGGGSGGGSGGGGSSDIVMSQSPDSLAVSLGERVTLNCKSSQSVLYSSNSKNYLAWYQQKPGQSPKLLIYWASTRESGVPDRFSGSGSGTDFTLTISSVQAEDVAVYYCQQYYSYPLSFGAGTKLELK

SEQ ID NO: 34 - protein, 246 aa, Homo sapiens
QVQLVQSGAEVKKPGASVKVSCKASGYTFTDHAIHWVRQAPGQRLEWMGYFSPGNDDFKYSQKFQGRVTITADTSASTAYMELSSLRSEDTAVYFCTRSLNMAYWGQGTLVTVSSGSTSGGGSGGGSGGGGSSDIVMTQSPDSLAVSLGERATINCKSSQSLLYSSNSKNYLAWYQQKPGQPPKLLIYWASTRESGVPDRFSGSGSGTDFTLTISSLQAEDVAVYYCQQPYSYPLSFGAGTKLELK

SEQ ID NO: 35 - protein, 554 aa, Homo sapiens
QVQLVQSGAEVVKPGASVKISCKASGYTFTDHAIHWVKQNPGQRLEWIGYFSPGNDDFKYNERFKGKATLTADTSASTAYVELSSLRSEDTAVYFCTRSLNMAYWGQGTLVTVSSGSTSGGGSGGGSGGGGSSDIVMSQSPDSLAVSLGERVTLNCKSSQSLLYSGNQKNYLAWYQQKPGQSPKLLIYWASARESGVPDRFSGSGSGTDFTLTISSVQAEDVAVYYCQQYYSYPLTFGAGTKLELKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 554 aa, Homo sapiens; identical to SEQ ID NO: 35

SEQ ID NO: 37 - protein, 554 aa, Homo sapiens
QVQLVQSGAEVVKPGASVKISCKASGYTFTDHAIHWVKQNPGQRLEWIGYFSPGNDDFKYSQKFQGKATLTADTSASTAYVELSSLRSEDTAVYFCTRSLNMAYWGQGTLVTVSSGSTSGGGSGGGSGGGGSSDIVMSQSPDSLAVSLGERVTLNCKSSQSVLYSSNSKNYLAWYQQKPGQSPKLLIYWASTRESGVPDRFSGSGSGTDFTLTISSVQAEDVAVYYCQQYYSYPLSFGAGTKLELKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 554 aa, Homo sapiens
QVQLVQSGAEVKKPGASVKVSCKASGYTFTDHAIHWVRQAPGQRLEWMGYFSPGNDDFKYSQKFQGRVTITADTSASTAYMELSSLRSEDTAVYFCTRSLNMAYWGQGTLVTVSSGSTSGGGSGGGSGGGGSSDIVMTQSPDSLAVSLGERATINCKSSQSLLYSSNSKNYLAWYQQKPGQPPKLLIYWASTRESGVPDRFSGSGSGTDFTLTISSLQAEDVAVYYCQQPYSYPLSFGAGTKLELKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 119 aa, Homo sapiens
QVQLVQSGAEVVKPGASVKISCKASGYTFTDHAIHWVKQNPGQRLEWIGYFSPGNDDFKYNERFKGKATLTADTSASTAYVELSSLRSEDTAVYFCTRSLNMAYWGQGTLVTVSSGSTS

SEQ ID NO: 40 - protein, 115 aa, Homo sapiens
SSDIVMSQSPDSLAVSLGERVTLNCKSSQSLLYSGNQKNYLAWYQQKPGQSPKLLIYWASARESGVPDRFSGSGSGTDFTLTISSVQAEDVAVYYCQQYYSYPLTFGAGTKLELK

SEQ ID NO: 41 - protein, 22 aa, Homo sapiens
MLLLVTSLLLCELPHPAFLLIP